(19) United States
(12) Patent Application Publication
(10) Pub. No.: US 2026/0199313 A1
(43) Pub. Date: Jul. 16, 2026

(54) METHODS OF TREATING ESSENTIAL TREMOR

(71) Applicant: Praxis Precision Medicines, Inc., Boston, MA (US)

(72) Inventors: Monique Giroux, Ashland, OR (US); Steven Petrou, Eltham (AU); Marcio Silva De Souza, Seabrook Island, NC (US)

(73) Assignee: Praxis Precision Medicines, Inc., Boston, MA (US)

(21) Appl. No.: 19/559,393

(22) Filed: Mar. 6, 2026

Related U.S. Application Data
(63) Continuation of application No. PCT/US2025/051351, filed on Oct. 16, 2025.
(60) Provisional application No. 63/708,215, filed on Oct. 16, 2024.
(60) Provisional application No. 63/717,025, filed on Nov. 6, 2024.
(60) Provisional application No. 63/781,117, filed on Mar. 31, 2025.
(60) Provisional application No. 63/824,633, filed on Jun. 16, 2025.

(51) Int. Cl. A61K 31/445; A61P 25/14

(52) U.S. Cl. CPC A61K 31/445; A61P 25/14

(57) ABSTRACT

The present disclosure relates to methods of treating essential tremor that comprise administering to a subject in need thereof compound of formula (I):

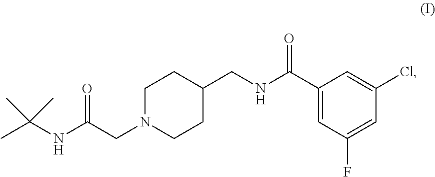 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 20 mg to about 60 mg; such that The Essential Tremor Rating Assessment Scale (TETRAS) performance score of said subject is decreased by at least 3 points following administration of the compound of formula (I), or a pharmaceutically acceptable salt thereof, as compared to the TETRAS performance score of said subject prior to the administration of the compound of formula (I), or a pharmaceutically acceptable salt thereof.

### RELATED APPLICATIONS

This application is a continuation of International Application No. PCT/US2025/051351, filed on Oct. 16, 2025; which claims priority to U.S. Provisional Patent Application No. 63/708,215, filed on Oct. 16, 2024; U.S. Provisional Patent Application No. 63/717,025, filed on Nov. 6, 2024; U.S. Provisional Patent Application No. 63/781,117, filed on Mar. 31, 2025; and U.S. Provisional Patent Application No. 63/824,633, filed on Jun. 16, 2025. The entire contents of each of the foregoing applications are hereby incorporated herein by reference.

### BACKGROUND

T-type calcium channels are low-voltage activated ion channels that mediate the influx of calcium into cells. Aberrant function or activity of these ion channels is associated with several diseases or conditions, including psychiatric disorders (e.g., mood disorders, such as a major depressive disorder), pain, tremor (e.g., essential tremor), epilepsy, or an epilepsy syndrome (e.g., absence seizures and juvenile myoclonic epilepsy).

In treating diseases or conditions related to aberrant function or activity of T-type calcium channels, the tolerability of potential pharmaceutical therapeutics is a factor to be considered. In investigations of new therapeutics, tolerability is a clinically-defined term that pertains to both patient (or study participant) and clinician (or investigator) input. The patient input includes their willingness to continue taking the therapeutic, and the clinician input includes their assessment of the severity and/or number of adverse effects associated with the therapeutic and a recommendation as to whether the patient should continue taking the therapeutic. Either party may decide that the drug is intolerable and thereby discontinue dosing.

Assessment of tolerability may also be based on the relationship between the adverse effects informing tolerability and the expected or perceived benefit. Accordingly, for a therapeutic or a therapeutic dose used for treating life-threatening diseases or conditions, more severe or an increased number of adverse events may be allowed before the therapeutic or the therapeutic dose may be designated as intolerable, as compared with a therapeutic or a therapeutic dose used for treating less serious diseases or conditions.

Essential tremor is a disorder with underlying aberrant T-type calcium channel function or activity, which may affect an individual's ability to function in daily life, but is typically not life-threatening. Essential tremor may, however, have a significant impact, on home life, work life, social life, or any combination thereof. For example, individuals with essential tremor may have difficulty performing daily activities, including certain tasks or jobs requiring motor skills, or feel embarrassed about their tremor. Essential tremor is one of the most common movement disorders, and is characterized by postural and/or kinetic tremors and, in some cases, impaired walking, along with some nonmotor symptoms. Propranolol (a beta blocker) and primidone (an anti-seizure medication), which are currently used for treating essential tremor, are characterized by serious side effects and are not effective in some patients. Accordingly, further methods of treating essential tremor are needed.

### SUMMARY

Accordingly, in some aspects, the present disclosure provides methods of treating essential tremor which comprise administering to a subject in need thereof, e.g., a human subject with essential tremor, a compound of formula (I):

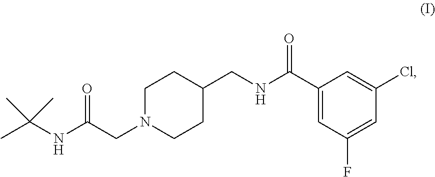 [SMILES: CC(C)(C)NC(=O)CN1CCC(CNC(=O)c2cc(F)cc(Cl)c2)CC1]

or a pharmaceutically acceptable salt thereof, wherein the compound of formula (I), or the pharmaceutically acceptable salt thereof, is administered to the subject once daily at a dose of about 60 mg to about 100 mg; such that The Essential Tremor Rating Assessment Scale (TETRAS) performance score of the subject is decreased following administration of the compound of formula (I), as compared to the TETRAS performance score of the subject prior to the administration of the compound of formula (I).

In some embodiments, the compound of Formula (I), or a pharmaceutically acceptable salt thereof, is administered to the subject at a dose of about 60 mg.

In some embodiments, the compound of formula (I), or a pharmaceutically acceptable salt thereof, is administered to the subject at a dose of about 100 mg.

In some embodiments, the compound of formula (I), or a pharmaceutically acceptable salt thereof, is administered to the subject following an initial titration period.

In some embodiments, the compound of formula (I), or a pharmaceutically acceptable salt thereof, is administered to the subject at a dose of about 60 mg following an initial titration period; wherein said initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period.

In some embodiments, the first time period, the second time period, the third time period and the fourth time period are each 7 days long.

In some embodiments, the compound of formula (I), or a pharmaceutically acceptable salt thereof, is administered to said subject at a dose of about 100 mg following an initial titration period; wherein the initial titration period comprises administering to the subject the compound of formula (I), or a pharmaceutically acceptable salt thereof, once daily at a dose of about 5 mg during a first time period; followed by a dose of about 10 mg during a second time period; followed by a dose of about 20 mg during a third time period; followed by a dose of about 40 mg during a fourth time period; followed by a dose of about 60 mg during a fifth time period, followed by a dose of about 80 mg during a sixth time period.

In some embodiments, the first time period, the second time period, the third time period, the fourth time period, the fifth time period and the sixth time period are each 7 days long.

In some embodiments, the compound of formula (I), or a pharmaceutically acceptable salt thereof, is administered to the subject for at least 8 weeks.

In some embodiments, the compound of formula (I), or a pharmaceutically acceptable salt thereof, is administered to the subject for at least 12 weeks.

In some embodiments, the compound of formula (I), or a pharmaceutically acceptable salt thereof, is administered to the subject for at least 14 weeks.

In some embodiments, the compound of formula (I), or a pharmaceutically acceptable salt thereof, is administered to the subject for at least 52 weeks.

In some embodiments, the TETRAS performance score comprises ADL subscore, and wherein the ADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of the subject prior to the administration of the compound of formula (I).

In some embodiments, the ADL subscore is decreased by at least about 2 points following at least 8 weeks of administration of the compound of formula (I), or a pharmaceutically acceptable salt thereof.

In some embodiments, the ADL subscore is decreased by at least about 3 points following at least 8 weeks of administration of the compound of formula (I), or a pharmaceutically acceptable salt thereof.

In some embodiments, the TETRAS performance score comprises mADL subscore, and wherein the mADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of said subject prior to the administration of the compound of formula (I).

In some embodiments, the mADL subscore is decreased by at least about 2 points following at least 8 weeks of administration of the compound of formula (I), or a pharmaceutically acceptable salt thereof.

In some embodiments, the mADL subscore is decreased by at least about 3 points following at least 8 weeks of administration of the compound of formula (I), or a pharmaceutically acceptable salt thereof.

In some embodiments, the mADL subscore is decreased by at least about 4 points following at least 8 weeks of administration of the compound of formula (I), or a pharmaceutically acceptable salt thereof.

In some embodiments, a subscore for at least one item assessed as a part of mADL subscale is decreased, wherein said item is selected from the group consisting of: item 3 (drinking from a glass); item 4 (hygiene); item 5 (dressing); item 6 (pouring); item 7 (carrying); item 8 (using keys); item 9 (writing); and item 11 (overall disability).

In some embodiments, the TETRAS performance score comprises mADL11 subscore, and wherein the mADL11 subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points, or at least about 10 points following administration of the compound of formula (I), as compared to the ADL subscore of said subject prior to the administration of the compound of formula (I).

In some embodiments, the mADL11 subscore is decreased by at least about 1 point following at least 8 weeks of administration of the compound of formula (I), or a pharmaceutically acceptable salt thereof.

In some embodiments, the mADL11 subscore is decreased by at least about 1.5 points following at least 8 weeks of administration of the compound of formula (I), or a pharmaceutically acceptable salt thereof.

In some embodiments, the mADL11 subscore is decreased by at least about 2 points following at least 8 weeks of administration of the compound of formula (I), or a pharmaceutically acceptable salt thereof.

In some embodiments, the mADL11 subscore is decreased by at least about 2.5 points following at least 8 weeks of administration of the compound of formula (I), or a pharmaceutically acceptable salt thereof.

In some embodiments, the subject does not have intention tremor.

In some embodiments, the subject is a human.

In some aspects, the present disclosure provides a method of treating essential tremor, the method comprising administering to a subject in need thereof compound of formula (I):

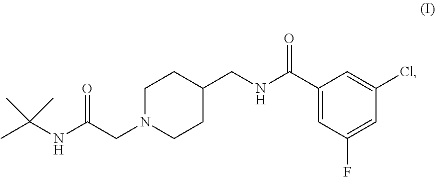 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; such that The Essential Tremor Rating Assessment Scale (TETRAS) modified Activities of Daily Living (mADL) subscore of the subject is decreased following administration of the compound of formula (I), or a pharmaceutically acceptable salt thereof, as compared to the mADL subscore of the subject prior to the administration of the compound of formula (I), or a pharmaceutically acceptable salt thereof.

In some embodiments, the compound of Formula (I), or a pharmaceutically acceptable salt thereof, is administered to said subject at a dose of about 60 mg. In some embodiments, the compound of formula (I), or a pharmaceutically acceptable salt thereof, is administered to said subject at a dose of about 100 mg.

In some embodiments, the compound of formula (I), or a pharmaceutically acceptable salt thereof, is administered to said subject following an initial titration period. In some embodiments, the initial titration period comprises administering to the subject the compound of formula (I), or a pharmaceutically acceptable salt thereof, once daily at a dose of about about 20 mg during a first time period; followed by a dose of about 40 mg during a second time period, followed by a dose of about 60 mg.

In some embodiments, the first time period and the second time period are each 7 days long. In some embodiments, the compound of formula (I), or a pharmaceutically acceptable salt thereof, is administered to the subject for at least 8 weeks. In some embodiments, the compound of formula (I), or a pharmaceutically acceptable salt thereof, is administered to the subject for at least 12 weeks, at least 14 weeks or at least 52 weeks.

In some embodiments, the mADL subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the mADL subscore of said subject prior to the administration of the compound of formula (I).

In some embodiments, the mADL subscore is decreased by at least about 2 points following at least 8 weeks of administration of the compound of formula (I), or a pharmaceutically acceptable salt thereof.

In some embodiments, the subject is a human.

In some aspects, the present disclosure provides a method of treating essential tremor, the method comprising administering to a subject in need thereof compound of formula (I):

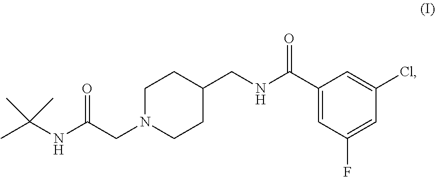 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; such that The Essential Tremor Rating Assessment Scale (TETRAS) modified Activities of Daily Living excluding performance subscale (mADL11) subscore of the subject is decreased following administration of the compound of formula (I), or a pharmaceutically acceptable salt thereof, as compared to the mADL11 subscore of the subject prior to the administration of the compound of formula (I), or a pharmaceutically acceptable salt thereof.

In some embodiments, the compound of Formula (I), or a pharmaceutically acceptable salt thereof, is administered to said subject at a dose of about 60 mg. In some embodiments, the compound of formula (I), or a pharmaceutically acceptable salt thereof, is administered to said subject at a dose of about 100 mg.

In some embodiments, the compound of formula (I), or a pharmaceutically acceptable salt thereof, is administered to said subject following an initial titration period.

In some embodiments, the initial titration period comprises administering to the subject the compound of formula (I), or a pharmaceutically acceptable salt thereof, once daily at a dose of about about 20 mg during a first time period; followed by a dose of about 40 mg during a second time period, followed by a dose of about 60 mg.

In some embodiments, the first time period and the second time period are each 7 days long. In some embodiments, the compound of formula (I), or a pharmaceutically acceptable salt thereof, is administered to the subject for at least 8 weeks. In some embodiments, the compound of formula (I), or a pharmaceutically acceptable salt thereof, is administered to the subject for at least 12 weeks, at least 14 weeks or at least 52 weeks.

In some embodiments, the mADL11 subscore is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL11 subscore of said subject prior to the administration of the compound of formula (I). In some embodiments, the mADL11 subscore is decreased by at least about 2 points following at least 8 weeks of administration of the compound of formula (I), or a pharmaceutically acceptable salt thereof.

In some embodiments, the subject is a human.

In some aspects, the present disclosure provides a method of treating essential tremor, the method comprising administering to a subject in need thereof compound of formula (I):

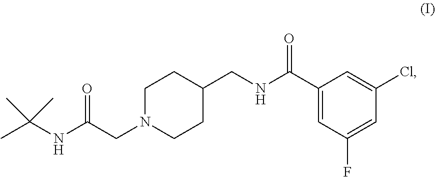 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; and wherein the subject has a diagnosis of essential tremor and does not have difficulty completing at least one task selected from the group consisting of pouring; drinking from a glass; picking up and/or reaching for small items; and using a smartphone. In some embodiments, the subject does not have difficulty completing at least two or more tasks selected from the group consisting of pouring; drinking from a glass; picking up and/or reaching for small items; and using a smartphone. In some embodiments, the subject does not have difficulty completing at least three or more tasks selected from the group consisting of pouring; drinking from a glass; picking up and/or reaching for small items; and using a smartphone. In some embodiments, the subject does not have difficulty completing any of pouring; drinking from a glass; picking up and/or reaching for small items; and using a smartphone.

In some aspects, the present disclosure provides a method of treating essential tremor, the method comprising administering to a subject in need thereof compound of formula (I):

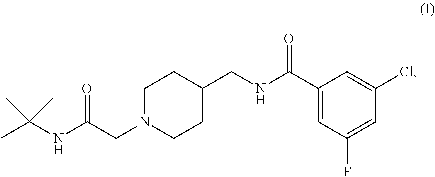 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; and wherein the subject has a diagnosis of essential tremor and does not have difficulty completing at least one task selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting. In some embodiments, the subject does not have difficulty completing at least two or more tasks selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting. In various embodiments, the subject does not have difficulty completing at least three or more, at least four or more, at least five or more, at least six or more, at least seven or more, at least eight or more, at least nine or more, at least 10 or more, at least 11 or more, or all twelve tasks selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting.

In various embodiments, assessment of difficulty is on the basis of mADL11, and neither difficulty completing left and right spirals nor difficulty completing handwriting is assessed.

In some embodiments, the subject does not have difficulty completing one or more, two or more, three or more, four or more, five or more, six or more, seven or more, eight or more, nine or more, ten or more, or any of the eleven measures of the mADL11.

In some aspects, the present disclosure provides a method of treating essential tremor, the method comprising administering to a subject in need thereof compound of formula (I):

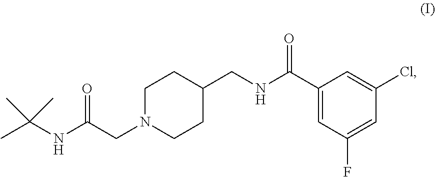 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; and wherein the subject has a diagnosis of essential tremor and has difficulty completing at least one task selected from the group consisting of pouring; drinking from a glass; picking up and/or reaching for small items; and using a smartphone. In some embodiments, the subject has difficulty completing at least two or more tasks selected from the group consisting of pouring; drinking from a glass; picking up and/or reaching for small items; and using a smartphone. In some embodiments, the subject has difficulty completing at least three or more tasks selected from the group consisting of pouring; drinking from a glass; picking up and/or reaching for small items; and using a smartphone. In some embodiments, the subject has difficulty completing any of pouring; drinking from a glass; picking up and/or reaching for small items; and using a smartphone.

In some aspects, the present disclosure provides a method of treating essential tremor, the method comprising administering to a subject in need thereof compound of formula (I):

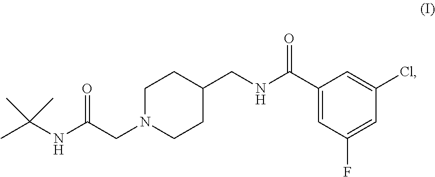 [SMILES: CC(C)(C)NC(=O)CN1CCC(CNC(=O)c2cc(F)cc(Cl)c2)CC1]

or a pharmaceutically acceptable salt thereof, wherein the compound of formula (I), or a pharmaceutically acceptable salt thereof, is administered to the subject once daily at a dose of about 60 mg to about 100 mg; and wherein the subject has a diagnosis of essential tremor and has difficulty completing at least one task selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting. In some embodiments, the subject has difficulty completing at least two or more tasks selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting. In various embodiments, the subject has difficulty completing at least three or more, at least four or more, at least five or more, at least six or more, at least seven or more, at least eight or more, at least nine or more, at least 10 or more, at least 11 or more, or all twelve tasks selected from the group consisting of speaking; feeding with a spoon; drinking from a glass; hygiene; dressing; pouring; carrying food trays, plates, or similar items; using keys; writing; working; overall disability with the most affected task; left and right spirals; and handwriting.

In various embodiments, assessment of difficulty is on the basis of mADL11, and neither difficulty completing left and right spirals nor difficulty completing handwriting is assessed.

In some embodiments, the subject has difficulty completing one or more, two or more, three or more, four or more, five or more, six or more, seven or more, eight or more, nine or more, ten or more, or any of the eleven measures of the mADL11.

### BRIEF DESCRIPTION OF THE FIGURES

FIG. 1 is a schematic listing items that are included in the TETRAS and TETRAS modified total score.

FIG. 2 is a schematic showing the design of the Phase 3 trial to compare the efficacy and safety of ulixacaltamide (Compound 1) with placebo in participants aged 18 to 80 years who have a diagnosis of ET.

FIG. 3 is a bar graph showing percent of responders willing to allow home visits and willing to use a mobile app and video calls.

FIG. 4 is a series of pie charts demonstrating that the essential tremor is underdiagnosed, with widespread impact on daily living.

FIG. 5 is a series of pie charts and bar graphs demonstrating essential tremor treatments and comorbidity burden.

FIG. 6 is a schematic of the phase 3 trial described in Example 1 which consisted of Study 1 (placebo-controlled parallel group study) and Study 2 (randomized-withdrawal study).

FIG. 7 is a schematic illustrating the analysis of the Study 1 data. The analysis illustrated a comparison of patients between ulixacaltamide and placebo after 56 days of intervention in the parallel design (PD) study.

FIG. 8 is a table describing baseline demographics of the participants of Study 1.

FIG. 9 is a series of graphs illustrating efficacy of ulixacaltamide in Study 1. The bar graph on the left in FIG. 9 shows that participants that were administered ulixacaltamide for 56 days in Study 1 (n=199) experienced a decrease in mADL11 score of 4.3 points, while the subjects administered placebo (n=233) experienced a decrease in mADL11 score of 1.7 points.

The graph on the right in FIG. 9 shows the results of least square means (LSM) analysis as it relates to changes in primary endpoint (mADL11 change from baseline) and secondary endpoints (rate of disease improvement through Day 84; PGI-C at day 56 and CGI-S at day 56).

The LSM analysis demonstrates that the results favor ulixacaltamide.

FIG. 10 is a graph illustrating a rapid and consistent response to ulixacaltamide administration through Day 84 in Study 1 as measured by change in mADL11 scores.

FIG. 11 is a graph illustrating a robust response to ulixacaltamide administration in Study 1 across different participant subgroups.

FIG. 12 is a schematic illustrating the analysis of the Study 2 data. This analysis illustrates that for patients exposed to ulixacaltamide in the randomized withdrawal (RW) study who improved by at least 3 points in the mADL11 scale, the proportion of those patients who maintained response after randomication staying on ulixacaltamide as compared to placebo.

FIG. 13 is a table describing baseline demographics of the participants of Study 2.

FIG. 14 is a series of graphs illustrating efficacy of ulixacaltamide in Study 2. The bar graph on the left in FIG. 14 shows that 55% of participants that were administered ulixacaltamide (n=40) and were stable responders, and 33% of participants that were administered placebo (n=40) maintained response, as defined by change in mADL11 score, following randomized withdrawal. The graph on the right in FIG. 14 also shows the results of LSM analysis, as it relates to secondary endpoints (rate of disease improvement from Day 56 through Day 84; PGI-C at day 84 and change in CGI-S from day 56 to Day 84) in placebo population (n=40) and ulixacaltabmide population (n=40) in Study 2, and favors ulixacaltamide. The LSM analysis demonstrates that the results favor ulixacaltamide.

FIG. 15 is a schematic illustrating the analysis of the Study 1 and Study 2 data. This analysis shows how the pooled group of patients receiving ulixacaltamide in both Study 1 and Study 2 and placebo patients from Study 1 after 56 days of intervention.

FIG. 16 is a series of graphs illustrating results of the analysis shown in FIG. 15. The bar graph on the left in FIG. 16 shows who participants that were administered ulixacaltamide in Study 1 and Study 2 (n=390) experienced a decrease in mADL11 score of 4.3 points, while the participants that were administered placebo in Study 1 (n=233) experienced a decrease in mADL11 score of 1.7 points. The graph on the right in FIG. 16 shows the results of LSM analysis as it relates to changes in primary endpoint (mADL11 change from baseline) at Day 14, Day 28, Day 49 and Day 56 in placebo population (n=233) and ulixacaltamide population (n=390). The LSM analysis demonstrates that the results favor ulixacaltamide.

FIG. 17 is a schematic illustrating the analysis of the Study 1 and Study 2 data. This analysis illustrates how patients receiving ulixacaltamide in the RW study compare to patients from the PD study after 56 days of intervention.

FIG. 18 is a series of graphs illustrating efficacy of ulixacaltamide based on the analysis illustrated in FIG. 17. The bar graph on the left in FIG. 18 shows that participants who were administered ulixacaltamide in Study 2 (blinded lead-in, n=191) experienced a decrease in mADL11 score of 4.3 points, while the subjects administered placebo in Study 1 (n=233) experienced a decrease in mADL11 score of 1.7 points. The graph on the right in FIG. 18 shows the results of LSM analysis as it relates to changes in primary endpoint (mADL11 change from baseline) at Day 14, Day 28, Day 49 and Day 56 in placebo population in Study 1 (n=233) and ulixacaltamide population in Study 2 (blinded lean-in, n=191). The LSM analysis demonstrates that the results favor ulixacaltamide.

FIG. 19 shows two tables describing disposition of participants in Study 1 and Study 2.

FIG. 20 shows a table with an overview of adverse events in the safety population.

FIG. 21 shows a table with an overview of most common TEAEs (>10%) in the safety population.

FIG. 22 is a series of schematics illustrating the results of an essential tremor neurologist survey.

FIG. 23 is a series of schematics illustrating the results of essential tremor patient surveys.

### DETAILED DESCRIPTION

### Methods of Treating Essential Tremor

In some aspects, the present disclosure provides methods of treating essential tremor that comprise administering to a subject in need thereof compound of formula (I):

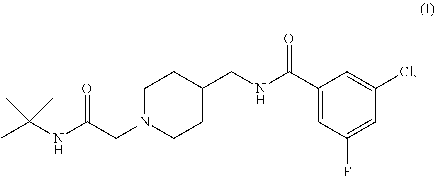 [SMILES: CC(C)(C)NC(=O)CN1CCC(CNC(=O)c2cc(F)cc(Cl)c2)CC1]

or a pharmaceutically acceptable salt thereof, wherein the compound of formula (I), or the pharmaceutically acceptable salt thereof, is administered to the subject once daily at a dose of about 60 mg to about 100 mg; such that The Essential Tremor Rating Assessment Scale (TETRAS) performance score of the subject is decreased following administration of the compound of formula (I), as compared to the TETRAS performance score of the subject prior to the administration of the compound of formula (I).

As used herein, a “subject” to which administration is contemplated includes, but is not limited to, a human and a non-human animal. A non-human animal may be a mammal, such as a primate (e.g., a cynomolgus monkey or a rhesus monkey), cattle, a pig, a horse, a sheep, a goat, a rodent (e.g., a mouse or a rat), a cat or a dog. In some embodiments, the subject is a non-human animal. In other embodiments, the subject is a human. In some embodiments, the subject is a male or female of any age group, such as a pediatric subject (e.g., infant, child, adolescent) or an adult subject (e.g., young adult, middle-aged adult or senior adult) The terms “human” and “patient” may be used interchangeably herein.

In some embodiments, the subject is a human. In some embodiments, the subject has essential tremor. In some embodiments, the subject does not have intention tremor. In some embodiments, the subject has intention tremor.

In some embodiments, the subject is a human and has been diagnosed with essential tremor. In some embodiments, the subject is a human, has been diagnosed with essential tremor and does not have difficulty completing at least one task, or at least two or more tasks selected from the group consisting of pouring; drinking from a glass; picking up and/or reaching for small items; and using a smartphone. In some embodiments, the subject is a human, has been diagnosed with essential tremor and does not have difficulty completing at least one task, or at least two or more tasks selected from the group consisting of speaking; feeding with a spoon; drinking from a glass; hygiene; dressing; pouring; carrying food trays, plates, or similar items; using keys; writing; working; left and right spirals; and handwriting.

In some embodiments, the subject is a human, has been diagnosed with essential tremor and has difficulty completing at least one task, or at least two or more tasks selected from the group consisting of pouring; drinking from a glass; picking up and/or reaching for small items; and using a smartphone. In some embodiments, the subject is a human, has been diagnosed with essential tremor and has difficulty completing at least one task, or at least two or more tasks selected from the group consisting of speaking; feeding with a spoon; drinking from a glass; hygiene; dressing; pouring; carrying food trays, plates, or similar items; using keys; writing; working; left and right spirals; and handwriting.

In some embodiments, the subject has a family history of essential tremor. In some embodiments, the subject has been diagnosed with essential tremor and has a family history of essential tremor.

In some embodiments, the subject has been diagnosed with essential tremor and has been treated with propranolol prior to being administered the compound of formula (I), or a pharmaceutically acceptable salt thereof. In some embodiments, the subject has been diagnosed with essential tremor and has not been treated with propranolol prior to being administered the compound of formula (I), or a pharmaceutically acceptable salt thereof. In some embodiments, the subject has been diagnosed with essential tremor and is administered the compound of formula (I), or a pharmaceutically acceptable salt thereof, in combination with propranolol.

As used herein, and unless otherwise specified, the terms “treat,” “treating” and “treatment” contemplate an action that occurs while a subject is suffering from the specified disease, disorder or condition, e.g., essential tremor, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

Methods of the present disclosure comprise administering to a subject in need thereof the compound of formula (I):

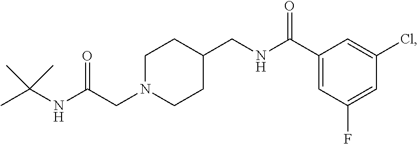 [SMILES: CC(C)(C)NC(=O)CN1CCC(CNC(=O)c2cc(F)cc(Cl)c2)CC1]

or a pharmaceutically acceptable salt (e.g., co-crystal) or solvate thereof.

As used herein, the term “pharmaceutically acceptable salt” refers to those salts which are, within sound medical judgment, suitable for use in contact with tissue of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compound of formula (I) include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include acetate, adipate, alginate, ascorbate, aspartate, benzenesulfonate, benzoate, besylate, bisulfate, borate, butyrate, camphorate, camphorsulfonate, citrate, cyclopentanepropionate, cyclamate, digluconate, dodecylsulfate, edisylate, ethanesulfonate, esylate, formate, fumarate, gentisate, glucoheptonate, glycerophosphate, gluconate, glucoronate, glutamate, glutarate, glycolate, hemisulfate, heptanoate, hexanoate, hippurate, hydroiodide, 2-hydroxy-ethanesulfonate, isethionate, ketoglutarate, lactobionate, lactate, laurate, lauryl sulfate, malate, maleate, malonate, mesylate, methanesulfonate, napadisylate, napsylate, 2-naphthalenesulfonate, nicotinate, nitrate, oleate, oxalate, oroate, palmitate, pamoate, pectinate, persulfate, 3-phenylpropionate, phosphate, picrate, pivalate, propionate, sebacate, stearate, succinate, sulfate, tartrate, thiocyanate, p-toluenesulfonate, undecanoate, valerate salts, stereoisomers thereof (e.g., enantiomers, diastereomers) and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In certain embodiments, compound of formula (I) is a hydrochloric acid salt, for example, compound of formula (II):

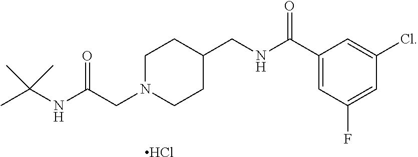 [SMILES: CC(C)(C)NC(=O)CN1CCC(CNC(=O)c2cc(F)cc(Cl)c2)CC1 ; Cl]

The compound of formula (I) may also be referred to as N-((1-(2-(tert-butylamino)-2-oxoethyl)piperidin-4-yl)methyl)-3-chloro-5-fluorobenzamide, while formula (II) may be referred to as N-((1-(2-(tert-butylamino)-2-oxoethyl)piperidin-4-yl)methyl)-3-chloro-5-fluorobenzamide hydrochloride.

In some embodiments, the compound of formula (I) or formula (II) may also be referred to herein as “ulixacaltamide” or “Compound 1”.

In some embodiments, methods of the present disclosure comprise administering to a subject in need thereof a crystalline form of the compound of formula (II), wherein the crystalline form exhibits an X-ray powder diffraction pattern comprising peaks at the following diffraction angles (2θ): 16.2±0.2, 17.4±0.2, and 26.6±0.2. In some embodiments, the crystalline form exhibits an X-ray powder diffraction pattern comprising peaks at the following diffraction angles (2θ): 11.5±0.2, 16.2±0.2, 17.4±0.2, 22.6±0.2, and 26.6±0.2. In some embodiments, the crystalline form exhibits an X-ray powder diffraction pattern comprising peaks at the following diffraction angles (2θ): 11.5±0.2, 16.2±0.2, 17.4±0.2, 18.3±0.2, 18.5±0.2, 19.2±0.2, 20.0±0.2, 22.6±0.2, 23.9±0.2, and 26.6±0.2. In some embodiments, the powder X-ray diffraction pattern was obtained using Cu Kα radiation. In some embodiments, the crystalline form has a melting point onset as determined by differential scanning calorimetry at about 226.6° C.

In some embodiments, methods of the present disclosure comprise administering to a subject in need thereof a crystalline form of the compound of formula (II), wherein the crystalline form exhibits an X-ray powder diffraction pattern comprising peaks at the following diffraction angles (2θ): 21.9±0.2, 18.5±0.2, and 17.8±0.2. In some embodiments, the crystalline form exhibits an X-ray powder diffraction pattern comprising peaks at the following diffraction angles (2θ): 21.9±0.2, 18.5±0.2, 17.8±0.2, 10.2±0.2, and 20.5±0.2. In some embodiments, the crystalline form exhibits an X-ray powder diffraction pattern comprising peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2, and 21.2±0.2. In some embodiments, the powder X-ray diffraction pattern was obtained using Cu Kα radiation. In certain embodiments, the crystalline form has a melting point onset as determined by differential scanning calorimetry at about 97.9, 131.6, 223.7, 83.8, 128.9, 168.9, or 224.4° C.

In some embodiments, compound of formula (I), compound of formula (II), or crystalline form of the compound of formula (II) is administered to a subject in need thereof in accordance with methods of the present disclosure as a part of a pharmaceutical composition also comprising a modified-release polymer. The term “modified-release polymer”, as used herein, refers to a polymer that is used in a composition (e.g., a tablet or a capsule) to modify the release rate of the drug upon administration to a subject. For example, a modified-release polymer is used to dissolve a drug over time in order to be released more slowly and steadily into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, HPMC (hydroxyl-propyl methylcellulose)), hydrophobic matrix polymers (e.g., ethyl cellulose, ethocel), or polyacrylate polymers (e.g., Eudragit RL100, Eudragit RS100).

In some embodiments, the pharmaceutical composition comprising the compound of formula (I), the compound of formula (II), or crystalline form of the compound of formula (II) comprises a diluent. The term “diluent”, as used herein, refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), and sugar alcohols (e.g., sorbitol, xylitol and mannitol)).

In some embodiments, the pharmaceutical composition comprising the compound of formula (I), the compound of formula (II), or crystalline form of the compound of formula (II) comprises a glidant. The term “glidant” as used herein refers to an excipient used to promote powder flow by reducing interparticle friction and cohesion. For example, glidants include fumed silica (e.g., colloidal silicon dioxide), talc, and magnesium carbonate.

In some embodiments, the pharmaceutical composition comprising the compound of formula (I), the compound of formula (II), or crystalline form of the compound of formula (II) comprises a lubricant. The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

In some embodiments, the pharmaceutical composition comprising the compound of formula (I), the compound of formula (II), or crystalline form of the compound of formula (II) comprises a coating. The term “coating” as used herein refers to an excipient, for example, to protect tablet ingredients from deterioration by moisture in the air and to make large or unpleasant-tasting tablets easier to swallow.

Methods of the present disclosure for treating essential tremor comprise administering to a subject in need thereof a titrated dose of the compound of formula (I), or a pharmaceutically acceptable salt thereof, such that the end or maintenance dosage (i.e., final dosage) exceeds an initial dosage or a dosage at which adverse events are likely to be experienced absent titration (a maximum tolerated dosage achieved without titration). As used herein, administering a tritrated dose refers to the practice of beginning with a low dosage and escalating to one or more higher dosages. Administration of titrated doses of the compound of formula (I), or a pharmaceutically acceptable salt thereof, is described, e.g., in WO 2021/222342 A1, the entire contents of which are hereby incorporated herein by reference.

In some embodiments, the compound of formula (I), or a pharmaceutically acceptable salt thereof, is administered to a subject in accordance with methods of the present disclosure at a final dose (e.g., 60 mg or 100 mg) after an initial titration period. The term “initial titration period”, when used in reference to administration of a final dose of the compound of formula (I), or a pharmaceutically acceptable salt thereof, e.g., a dose of about 60 mg or about 100 mg, refers to a period of time during which escalating doses which are lower than the final dose of the compound of formula (I), or a pharmaceutically acceptable salt thereof, are administered to a subject until the final dose of the compound of formula (I), or a pharmaceutically acceptable salt thereof, is achieved, e.g., a dose of about about 60 mg or about 100 mg.

For example, an initial titration period may comprise administration to a subject of escalating doses of the compound of formula (I), or a pharmaceutically acceptable salt thereof, until a dose of about 60 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg and a dose of about 40 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the first, second, third and fourth time periods are each 7 days long. In some embodiments, the compound of formula (I), or a pharmaceutically acceptable salt thereof, is administered once daily.

In some embodiments, an initial titration period may comprise administration to a subject of escalating doses of the compound of formula (I), or a pharmaceutically acceptable salt thereof, until a dose of about 60 mg is achieved over, e.g., a period of 1 week, 2 weeks, 3 weeks or 4 weeks. In some embodiments, the compound of formula (I), or a pharmaceutically acceptable salt thereof, is administered to a subject at a daily dose of about 20 mg for 7 days, followed by administration at a daily dose of about 40 mg for 7 days, followed by administration at a daily dose of about 60 mg.

In another example, an initial titration period may comprise administration to a subject of escalating doses of the compound of formula (I), or a pharmaceutically acceptable salt thereof, until a dose of about 80 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg, a dose of about 40 mg, and a dose of about 60 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period; followed by a dose of about 60 mg during the fifth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the fifth time period may be about 1 day, about 2 days, about 3 days, about 4 days, about 5 days, about 6 days or about 7 days long. In some embodiments, the first, second, third, fourth and fifth time periods are each 7 days long. In some embodiments, the compound of formula (I), or a pharmaceutically acceptable salt thereof, is administered once daily.

In yet another example, an initial titration period may comprise administration to a subject of escalating doses of the compound of formula (I), or a pharmaceutically acceptable salt thereof, until a dose of about 100 mg is achieved. In some embodiments, an initial titration period may comprise administration to a subject of one or more doses of the compound of formula (I), or a pharmaceutically acceptable salt thereof, selected from the group consisting of: a dose of about 5 mg, a dose of about 10 mg, a dose of about 20 mg, a dose of about 40 mg, a dose of about 60 mg and a dose of about 80 mg. In some embodiments, an initial titration period may comprise administration to a subject of a dose of the compound of formula (I), or a pharmaceutically acceptable salt thereof, of about 5 mg during a first time period; followed by a dose of about 10 mg during a second time period; followed by a dose of about 20 mg during a third time period; followed by a dose of about 40 mg during a fourth time period; followed by a dose of about 60 mg during the fifth time period; followed by a dose of about 80 mg during the sixth time period. In some embodiments, the first time period may be about 1 day, about 2 days, about 3 days, about 4 days, about 5 days, about 6 days or about 7 days long. In some embodiments, the second time period may be about 1 day, about 2 days, about 3 days, about 4 days, about 5 days, about 6 days or about 7 days long. In some embodiments, the third time period may be about 1 day, about 2 days, about 3 days, about 4 days, about 5 days, about 6 days or about 7 days long. In some embodiments, the fourth time period may be about 1 day, about 2 days, about 3 days, about 4 days, about 5 days, about 6 days or about 7 days long. In some embodiments, the fifth time period may be about 1 day, about 2 days, about 3 days, about 4 days, about 5 days, about 6 days or about 7 days long. In some embodiments, the sixth time period may be about 1 day, about 2 days, about 3 days, about 4 days, about 5 days, about 6 days or about 7 days long. In some embodiments, the first, second, third, fourth, fifth and sixth time periods are each 7 days long. In some embodiments, the compound of formula (I), or a pharmaceutically acceptable salt thereof, is administered once daily.

In certain aspects of all embodiments disclosed herein, the time period of administration, such as the first, second, third, fourth, fifth, or sixth time period, may range from about 3 days to about 1 year. For example, the first, second, third, fourth, fifth and/or sixth time period may be 3 days, 4 days, 5 days, 6 days, 7 days, 8 days, 9 days, 10 days, 11 days, 12 days, 13 days, 14 days, 15 days, 16 days, 21 days, 28 days, 35 days, 42 days, 49 days, 56 days, 63 days, 70 days, 77 days, 84 days, 91 days, 98 days, 105 days, 112 days, 119 days, 126 days, 133 days, 140 days, 147 days, 154 days, 161 days, 168 days, 175 days, 182 days, 189 days, 196 days, 203 days, 210 days, 217 days, 224 days, 231 days, 238 days, 245 days, 252 days, 259 days, 266 days, 273 days, 280 days, 287 days, 294 days, 301 days, 308 days, 315 days, 322 days, 329 days, 336 days, 343 days, 350 days, 357 days, 364 days, or 1 year long. In certain aspects of the embodiments disclosed herein, the time period of administration, such as the seventh time period, may range from about 3 to about 16 days, such as, for example, 3, 4, 5, 6, 7, 8, 9, 10, 11, 12, 13, 14, 15, or 16 days. In other aspects of the embodiments disclosed herein, of the time periods, e.g., the first, second, third, fourth, fifth, sixth, or seventh time period of administration may be greater than 14 days.

In certain aspects, the dosage increase relative to the prior dose is not more than 40 mg per day. For instance, in certain embodiments, the second dose is increased no more than 40 mg per day relative to the first dose, and in certain aspects, the third dose is increased no more than 40 mg per day relative to the second dose. In other aspects, the dosage increase relative to the prior dose is not more than 20 mg per day. For instance, in certain embodiments, the second dose is increased no more than 20 mg per day relative to the first dose, and in certain embodiments, the third dose is increased no more than 20 mg per day relative to the second dose. In some embodiments, the third dose is the maintenance, or final dose.

In accordance with methods of the present disclosure, a compound of formula (I), or a pharmaceutically acceptable salt thereof, may be administered to a subject once, twice, three times daily at a dose of up to about 120 mg (e.g., from about 5 mg to about 120 mg, from about 10 mg to about 120 mg, from about 15 mg to about 120 mg, from about 20 mg to about 120 mg, from about 40 mg to about 120 mg, from about 5 mg to about 100 mg, from about 10 mg to about 100 mg, from about 15 mg to about 100 mg, from about 20 mg to about 100 mg, from about 40 mg to about 100 mg, from about 5 mg to about 80 mg, from about 10 mg to about 80 mg, from about 15 mg to about 80 mg, from about 20 mg to about 80 mg, from about 40 mg to about 80 mg, from about 5 mg to about 60 mg, from about 10 mg to about 60 mg, from about 15 mg to about 60 mg, from about 20 mg to about 60 mg, or from about 40 mg to about 60 mg). In some embodiments, the compound of formula (I), or a pharmaceutically acceptable salt thereof, is administered to the subject once daily.

In some embodiments, methods of treating essential tremor in a subject in need thereof in accordance with methods of the present disclosure comprise:
(a) administering to the subject once daily during a first time period (e.g., 3, 4, 5, 6, 7, 8, or 9 days), 5 mg of the compound of formula (I) or a pharmaceutically acceptable salt thereof (e.g., the compound of formula (II));
(b) administering to the subject once daily during a second time period (e.g., 3, 4, 5, 6, 7, 8, or 9 days), 10 mg of the compound of formula (I) or a pharmaceutically acceptable salt thereof (e.g., the compound of formula (II)); and
(c) administering to the subject once daily during a third time period (e.g., 3, 4, 5, 6, 7, 8, or 9 days), 20 mg of the compound of formula (I) or a pharmaceutically acceptable salt thereof (e.g., the compound of formula (II)). In some embodiments, a compound of formula (I), or a pharmaceutically acceptable salt thereof, is administered to the subject once daily.

In some embodiments, methods of treating essential tremor in a subject in need thereof in accordance with methods of the present disclosure comprise:
(a) administering to the subject during a first time period (e.g., 3, 5, 6, 7, 8, or 9 days), 20-40 mg per day of the compound of formula (I) or a pharmaceutically acceptable salt thereof (e.g., the compound of formula (II));
(b) administering to the subject during a second time period (e.g., 3, 5, 6, 7, 8, or 9 days), 20-60 mg per day of the compound of formula (I) or a pharmaceutically acceptable salt thereof (e.g., the compound of formula (II)); and
(c) administering to the subject during a third period (e.g., 3, 4, 5, 6, 7, 8, or 9 days), 20-80 mg per day of the compound of formula (I) or a pharmaceutically acceptable salt thereof (e.g., the compound of formula (II)).

In some embodiments, the method further comprises (d) administering to the subject during a fourth time period (e.g., 3, 4, 5, 6, 7, 8, or 9 days), 20-100 mg per day of the compound of formula (I) or a pharmaceutically acceptable salt thereof (e.g., the compound of formula (II)).

In other embodiments, the method further comprises (e) administering to the subject during a fifth time period (e.g., 3, 4, 5, 6, 7, 8, or 9 days), 20-120 mg per day of the compound of formula (I) or a pharmaceutically acceptable salt thereof (e.g., the compound of formula (II)).

In certain embodiments, the method further comprises (f) administering to the subject during a sixth time period (e.g., 3, 4, 5, 6, 7, 8, 9, 10, 11, 12, 13, 14, 15, or 16 days), 20-120 mg per day of the compound of formula (I) or a pharmaceutically acceptable salt thereof (e.g., the compound of formula (II)).

In certain embodiments, the method further comprises (g) administering to the subject during a seventh time period (e.g., 3, 4, 5, 6, 7, 8, 9, 10, 11, 12, 13, or 14 or more days), 20-120 mg per day of the compound of formula (I) or a pharmaceutically acceptable salt thereof (e.g., the compound of formula (II)) as needed.

A physician may elect not to continue to escalate the dose of the compound of formula (I) or a pharmaceutically acceptable salt thereof, such that there may be less than the seven time periods described above. For example, there may be only 1, 2, 3, 4, 5, or 6 time periods of escalating dosages needed to achieve a desired therapeutic effect. In some embodiments, methods of the present disclosure comprise 2, 3 or 4 time periods of escalating dosages. In some embodiments, methods of the present disclosure comprise 5 time periods of escalating dosages. In some embodiments, methods of the present disclosure comprise 7 time periods of escalating dosages.

In some embodiments, methods of the present disclosure comprise: (a) administering to the subject during a first time period ranging from about 3 to about 9 days, about 5 mg to about 40 mg per day of a compound of formula (I) or a pharmaceutically acceptable salt thereof, such as 20 mg or 40 mg per day; (b) administering to the subject during a second time period ranging from about 3 to about 9 days, about 10 mg to about 100 mg per day of the compound of formula (I) or a pharmaceutically acceptable salt thereof, such as 40 mg, 60 mg, or 80 mg per day; and (c) administering to the subject during a third time period ranging from about 3 to about 9 days, about 20 mg to about 120 mg per day of the compound of formula (I) or a pharmaceutically acceptable salt thereof, such as 60 mg, 80 mg, 100 mg, or 120 mg per day.

In some embodiments, methods of the present disclosure comprise: (a) administering a first dose of about 20 mg to about 40 mg per day of a compound of formula (I) or a pharmaceutically acceptable salt thereof to a subject once daily during a first time period; (b) increasing the amount of the compound in the first dose and administering one or more increased doses of the compound to the subject to arrive at a maximum titrated dose of about 80 mg to about 120 mg per day; and (c) administering the maximum titrated dose to the subject once daily as needed.

In related embodiments, the dosage of the compound of formula (I) may be adjusted upward or downward in 1, 2, 3, 4, 5, 10, 15, 20 mg increments as deemed necessary by a physician depending on a subject's response to the prior dosages of compound of formula (I).

In various embodiments of the disclosure, methods disclosed herein comprise: (a) administering a first dose of 20 mg per day during a first time period of 3 days, (b) administering a second dose of 40 mg per day during a second time period 3 days, (c) administering a third dose of 60 mg per day during a third time period of 7 days; (d) administering a fourth dose of 80 mg per day during a fourth time period of 7 days; (e) administering a fifth dose of 100 mg per day during a fifth time period of 7 days; and (f) thereafter administering a sixth dose of 120 mg per day as needed. In various other embodiments, the methods disclosed herein comprise (a) administering a first dose of 20 mg per day for a first time period of 3 days, (b) administering a second dose of 40 mg per day for a second time period of 3 days, (c) administering a third dose of 80 mg per day for a third time period of 3 days; and (d) administering a fourth dose of 120 mg per day as needed.

In certain embodiments there is disclosed a method of treating a disease or condition relating to aberrant function or activity of T-type calcium channels in a subject in need thereof comprising (a) administering to the subject during a first time period of 3 days about 20 mg per day of a compound of formula (I) or a pharmaceutically acceptable salt thereof, (b) administering to the subject during a second time period of about 3 days about 40 mg per day of the compound; (c) administering to the subject during a third time period of about 3 days about 60 mg per day of the compound; (d) administering to the subject during a fourth time period of about 3 days about 80 mg per day of the compound; (e) administering to the subject during a fifth time period of about 3 days about 100 mg per day of the compound; and (f) administering to the subject during a sixth time period of time about 120 mg per day of the compound. Instead of every 3 days, the doses can also be administered every 4, 5, or 6 days.

Also disclosed herein is a method of treating essential tremor in a subject in need thereof, comprising: (a) administering to the subject during a first time period of 7 days about 20 mg per day of a compound of formula (I) or a pharmaceutically acceptable salt thereof, (b) administering to the subject during a second time period of about 7 days about 40 mg per day of the compound; (c) administering to the subject during a third time period of about 7 days about 60 mg per day of the compound; (d) administering to the subject during a fourth time period of about 7 days about 80 mg per day of the compound; (e) administering to the subject during a fifth time period of about 7 days about 100 mg per day of the compound; and (f) administering to the subject during a sixth period of time about 120 mg per day of the compound.

In some embodiment, methods of the present disclosure comprise: (a) administering to the subject during a first time period of 3 days about 40 mg per day of a compound of formula (I) or a pharmaceutically acceptable salt thereof, (b) administering to the subject during a second time period of about 3 days about 80 mg per day of the compound; and (c) administering to the subject during a third period about 120 mg per day of the compound. Instead of every 3 days, the doses can also be administered every 4, 5, or 6 days.

Also disclosed herein is a method of treating a disease or condition relating to aberrant function or activity of a T-type calcium channel in a subject in need thereof comprising (a) administering to the subject for a first period of 7 days about 40 mg per day of a compound of formula (I) or a pharmaceutically acceptable salt thereof, (b) administering to the subject for a second period of about 7 days about 80 mg per day of the compound; and (c) administering to the subject for a third period about 120 mg per day of the compound.

In certain embodiments there is disclosed a method of treating a disease or condition relating to aberrant function or activity of a T-type calcium channel in a subject in need thereof comprising (a) administering to the subject for a first period of 7 days about 20 mg per day of a compound of formula (I) or a pharmaceutically acceptable salt thereof, (b) administering to the subject for a second period of about 7 days about 40 mg per day of the compound; (c) administering to the subject for a third period of about 7 days about 60 mg per day of the compound; (d) administering to the subject for a fourth period of about 7 days about 80 mg per day of the compound; (e) administering to the subject for a fifth period of about 7 days about 100 mg per day of the compound; (f) administering to the subject for a sixth period of about 14 days about 120 mg per day of the compound; and (g) thereafter administering to the subject about 1-120 mg per day of the compound as needed.

A physician may choose to stop escalating the dose of compound of formula (I) or a pharmaceutically acceptable salt thereof once the subject has demonstrated a desired therapeutic effect. At this point, the physician may elect for the subject to continue the dose that the subject has been taking to achieve the desired therapeutic effect, or may elect for the subject to lower the dose of the compound of formula (I), or a pharmaceutically acceptable salt thereof, in order to maintain the desired therapeutic effect, reduce side effects, or both. Achieving a desired therapeutic effect may be observed by improvements in, for example, the subject's TETRAS performance score, mADL score, mADL11 score, accelerometer performance score, or Archimedes spiral task test.

In some embodiments, methods of treating essential tremor provided by the present disclosure comprise administering to a subject in need thereof a compound of formula (I), or a pharmaceutically acceptable salt thereof, such that The Essential Tremor Rating Assessment Scale (TETRAS) performance score of the subject is decreased following administration of the compound of formula (I), or a pharmaceutically acceptable salt thereof, as compared to the TETRAS performance score of the subject prior to the administration of the compound of formula (I), or a pharmaceutically acceptable salt thereof. TETRAS was developed to quantify essential tremor severity and its impact on activities of daily living, as described in Elble et al., J. Neurol Neuromedicine (2016), 1(4):34-38, the entire contents of which are hereby incorporated herein by reference. In certain embodiments, methods of treating essential tremor in a subject in need thereof, may result in a change in TETRAS performance score of the subject. The TETRAS performance score is assessed using the TETRAS scale, which consists of two sections: the Performance Subscale (TETRAS-PS) and the Activities of Daily Living (TETRAS-ADL) subscale. TETRAS-PS section includes 9 items as listed below:

| Item No. | Item Description |
|---|---|
| 1 | Head tremor |
| 2 | Face tremor |
| 3 | Voice tremor |
| 4 | Kinetic tremor |
| 5 | Lower limb tremor |
| 6 | Spiral drawing |
| 7 | Handwriting |
| 8 | Dot approximation task |
| 9 | Standing |

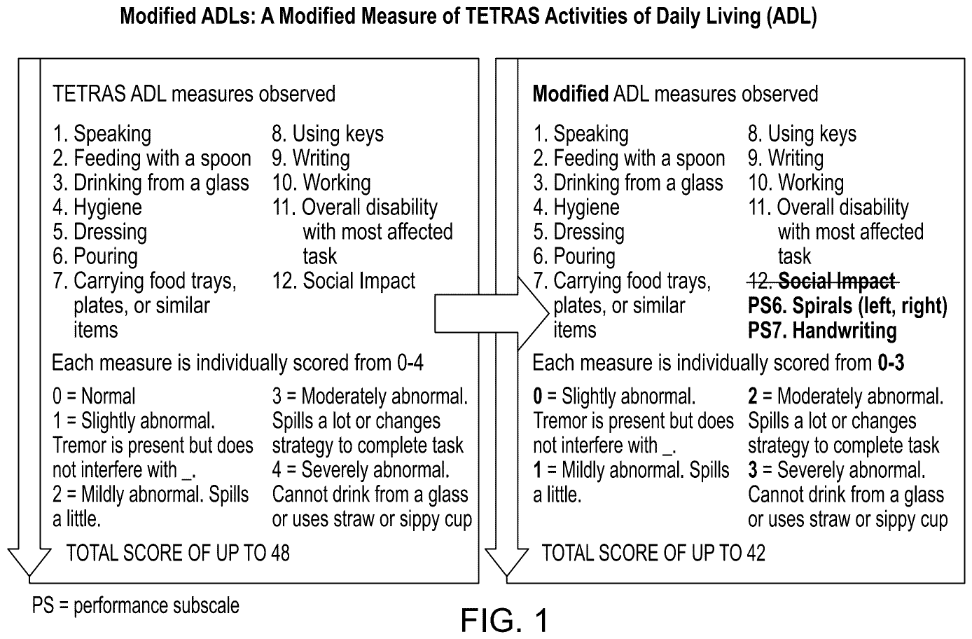

The TETRAS-ADL, or ADL subscale is a 12-item assessment of typical daily activities that are impacted by tremor. As shown in FIG. 1, activities in the TETRAS-ADL subscale are assessed in the following functional domains:

| Item No. | Item Description |
|---|---|
| 1 | Speaking |
| 2 | Feeding (e.g., with a spoon) |
| 3 | Drinking from a glass |
| 4 | Hygiene (e.g., personal hygiene) |
| 5 | Dressing |
| 6 | Pouring |
| 7 | Carrying (e.g., food trays, plates or similar) |
| 8 | Using keys |
| 9 | Writing |
| 10 | Working |
| 11 | Overall disability with most affected task |
| 12 | Social impact |

The impact to each function is rated on a 5-point Likert scale from 0 to 4. The full ADL subscale score (ADL subscore) is calculated as the sum of all 12 items and ranges from 0 to 48.

TETRAS modified ADL subscore (mADL) is a a composite sum of items 1 to 11 in TETRAS-ADL subscale (omitting item 12 social impact) and items 6 and 7 of the TETRAS-PS subscale (spiral drawing and handwriting). Scoring in mADL is adjusted, such that response options 0 and 1 are combined into a single response of “0=Normal.”, and scores of 2, 3, and 4 are adjusted to 1, 2, and 3, respectively. The TETRAS modified total score is the sum of the adjusted TETRAS-ADL and TETRAS PS items, having a possible range of 0 to 42, where larger values represent increased direct tremor impact to activities of daily living. The items that are included in the TETRAS-modified total score are illustrated in FIG. 1 and are shown below.

TETRAS mADL subscore is a composite sum of the following items:

| | |
|---|---|
| Item No. | Item Description |

Items from TETRAS-ADL subscale

| | |
|---|---|
| 1 | Speaking |
| 2 | Feeding (e.g., with a spoon) |
| 3 | Drinking from a glass |
| 4 | Hygiene (e.g., personal hygiene) |
| 5 | Dressing |
| 6 | Pouring |
| 7 | Carrying (e.g., food trays, plates or similar) |
| 8 | Using keys |
| 9 | Writing |
| 10 | Working |
| 11 | Overall disability with most affected task |

Items from TETRAS-PS subscale

| | |
|---|---|
| 6 | Spiral drawing |
| 7 | Handwriting. |

TETRAS modified ADL excluding performance subscale (mADL11) subscore is a composite sum of items 1 to 11 of the TETRAS-ADL subscale (omitting item 12 social impact score and items 6 and 7 of TETRAS-PS subscale). Scoring of each item is transformed to a 4-point scale from 0 to 3 with total score range from 0 to 33.

TETRAS mADL11 subscore is a composite sum of the following items:

Items from TETRAS-ADL subscale

| Item No. | Item Description |
|---|---|
| 1 | Speaking |
| 2 | Feeding (e.g., with a spoon) |
| 3 | Drinking from a glass |
| 4 | Hygiene (e.g., personal hygiene) |
| 5 | Dressing |
| 6 | Pouring |
| 7 | Carrying (e.g., food trays, plates or similar) |
| 8 | Using keys |
| 9 | Writing |
| 10 | Working |
| 11 | Overall disability with most affected task. |

In some embodiments, a decrease in TETRAS performance score, e.g., one or more of ADL, mADL or mADL11, is indicative of a desired therapeutic effect. In some embodiments, a decrease in TETRAS performance score, e.g., one or more of ADL, mADL or mADL11, is indicative of an improvement in the symptoms of essential tremor.

In some embodiments, methods of treating essential tremor in a subject in need thereof provided by the present disclosure may result in a decrease in TETRAS performance score in the subject. Thus, in some embodiments, TETRAS performance score of a subject is decreased following administration of the compound of formula (I), or a pharmaceutically acceptable salt, to the subject in accordance to methods of the present disclosure, as compared to TETRAS performance score of the subject prior to administration of the compound of formula (I), or a pharmaceutically acceptable salt thereof.

In some embodiments, the TETRAS performance score comprises ADL subscore, and the ADL subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the ADL subscore of the subject prior to the administration of the compound of formula (I). In some embodiments, the ADL subscore is decreased by at least about 2 points following at least 8 weeks of administration of the compound of formula (I), or a pharmaceutically acceptable salt thereof to the subject. In some embodiments, the ADL subscore is decreased by at least about 3 points following at least 8 weeks of administration of the compound of formula (I), or a pharmaceutically acceptable salt thereof to the subject.

In some embodiments, the TETRAS performance score comprises mADL subscore, and the mADL subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the mADL subscore of the subject prior to the administration of the compound of formula (I). In some embodiments, the mADL subscore is decreased by at least about 2 points following at least 8 weeks of administration of the compound of formula (I), or a pharmaceutically acceptable salt thereof to the subject. In some embodiments, the mADL subscore is decreased by at least about 3 points following at least 8 weeks of administration of the compound of formula (I), or a pharmaceutically acceptable salt thereof to the subject. In some embodiments, the mADL subscore is decreased by at least about 2 points following at least 8 weeks of administration of the compound of formula (I), or a pharmaceutically acceptable salt thereof, to the subject.

In some embodiments, the TETRAS performance score comprises mADL11 subscore, and the mADL11 subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the mADL11 subscore of the subject prior to the administration of the compound of formula (I). In some embodiments, the mADL11 subscore is decreased by at least about 2 points following at least 8 weeks of administration of the compound of formula (I), or a pharmaceutically acceptable salt thereof to the subject. In some embodiments, the mADL11 subscore is decreased by at least about 3 points following at least 8 weeks of administration of the compound of formula (I), or a pharmaceutically acceptable salt thereof to the subject. In some embodiments, the mADL subscore is decreased by at least about 2 points following at least 8 weeks of administration of the compound of formula (I), or a pharmaceutically acceptable salt thereof, to the subject.

In some embodiments, in accordance with methods of the present disclosure, administration of the compound of formula (I), or a pharmaceutically acceptable salt thereof, to a subject in need thereof for treating essential tremor leads to a decrease in a subscore for at least one item assessed as a part of mADL subscale. In some embodiment, the item is selected from the group consisting of: item 3 (drinking from a glass); item 4 (hygiene); item 5 (dressing); item 6 (pouring); item 7 (carrying); item 8 (using keys); item 9 (writing); and item 11 (overall disability).

In some embodiments, the TETRAS performance score comprises mADL11 subscore, and the mADL11 subscore of a subject is decreased by at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points, or at least about 10 points, following administration of the compound of formula (I), as compared to the mADL11 subscore of the subject prior to the administration of the compound of formula (I).

In some embodiments, the mADL11 subscore is decreased by at least about 2 points following at least 8 weeks of administration of the compound of formula (I), or a pharmaceutically acceptable salt thereof. In some embodiments, mADL11 subscore is decreased by at least about 2.5 points following at least 8 weeks of administration of the compound of formula (I), or a pharmaceutically acceptable salt thereof. In some embodiments, mADL11 subscore is decreased by at least about 3 points following at least 8 weeks of administration of the compound of formula (I), or a pharmaceutically acceptable salt thereof. In some embodiments, mADL11 subscore is decreased by at least about 3.5 points following at least 8 weeks of administration of the compound of formula (I), or a pharmaceutically acceptable salt thereof. In some embodiments, mADL11 subscore is decreased by at least about 4 points following at least 8 weeks of administration of the compound of formula (I), or a pharmaceutically acceptable salt thereof.

In some embodiments, methods of treating a disease or condition relating to aberrant function or activity of a T-type calcium channel, e.g., essential tremor, in a subject in need thereof may result in a change, e.g., a decrease, in TETRAS performance score of the subject. In some embodiments, methods of treating a disease or condition relating to aberrant function or activity of a T-type calcium channel, e.g., essential tremor, in a subject in need thereof, may result in a change, e.g., a decrease, in ADL performance subscore score of the subject. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore of the subject. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score, e.g., mADL11, of the subject of at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater. In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least about 0.5 points, at least about 1 point, at least about 1.5 points, at least about 2 points, at least about 2.5 points, at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least 10 points when compound of formula (I) is administered at a dose of about 60 mg once daily after an initial titration period.

The Patient Global Impression—Change (PGI-C) assesses subject's improvement (or worsening). The subject is required to assess his or her condition relative to Baseline (Pre-dose on Day 1) on a 7-point scale which include the following scores: 1 (very much improved), 2 (much improved), 3 (minimally improved), 4 (no change), 5 (worse), 6 (much worse) and 7 (very much worse). In all cases, the assessment is to be made independently of whether the participant believes the improvement/worsening is drug-related or not.

In some embodiments, in accordance with methods of the present disclosure, administration of the compound of formula (I), or a pharmaceutically acceptable salt thereof, to a subject in need thereof for treating essential tremor leads to a PGI-C score of 1, 2, 3 or 4.

In some embodiments, in accordance with methods of the present disclosure, administration of the compound of formula (I), or a pharmaceutically acceptable salt thereof, to a subject in need thereof for treating essential tremor leads to an improved PGI-C score. In some embodiments, in accordance with methods of the present disclosure, administration of the compound of formula (I), or a pharmaceutically acceptable salt thereof, to a subject in need thereof for treating essential tremor leads to a PGI-C score of 1, 2 or 3.

The Clinical Global Impression—Severity (CGI-S) assesses clinician's impression of a subject's current illness state. In determining CGI-S, a clinician uses his or her total clinical experience with this patient population and rates the current severity of the subject's ET on a 7-point scale from 1 (normal, not at all ill) to 7 (among the most extremely ill patients).

In some embodiments, in accordance with methods of the present disclosure, administration of the compound of formula (I), or a pharmaceutically acceptable salt thereof, to a subject in need thereof for treating essential tremor leads to a decrease in CGI-S score of the subject. In some embodiments, in accordance with methods of the present disclosure, administration of the compound of formula (I), or a pharmaceutically acceptable salt thereof, to a subject in need thereof for treating essential tremor leads to a decrease in CGI-S score of at least about 0.1 point, at least about 0.2 points, at least about 0.3 points, at least about 0.4 points, at least about 0.5 points, at least about 0.6 points, at least about 0.7 points, at least about 0.8 points, at least about 0.9 points, at least about 1.0 points, at least about 1.1 points, at least about 1.2 points, at least about 1.3 points, at least about 1.4 points, at least about 1.5 points, at least about 1.6 points, at least about 1.7 points, at least about 1.8 points, at least about 1.9 points, or at least about 2.0 points.

In an embodiment, in accordance with methods of the present disclosure, administration of the compound of formula (I), or a pharmaceutically acceptable salt thereof, to a subject in need thereof for treating essential tremor leads to a decrease in CGI-S score from 7, 6, 5, 4, 3, or 2 at any timepoint by any amount that leads to a lower CGI-S score. By way of non-limiting example, a CGI-S score of 7 may be decreased to 6, 5, 4, 3, 2, or 1. Likewise, for example, a CGI-S score of 6 may be decreased to 5, 4, 3, 2, or 1. In other embodiments, a CGI-S score of 5 may be decreased to 4, 3, 2, or 1; a CGI-S score of 4 may be decreased to 3, 2, or 1. A CGI-S score of 3 may be decreased to 2 or 1, and a CGI-S score of 2 may be decreased to 1.

In some embodiments, a subject being treated with the compound of formula (I), or a pharmaceutically acceptable salt thereof, in accordance with methods of the present disclosure, does not experience serious adverse events. In some embodiments, a subject being treated with the compound of formula (I), or a pharmaceutically acceptable salt thereof, in accordance with methods of the present disclosure, experiences mild to moderate adverse events.

In some embodiments, the compound of formula (I), or a pharmaceutically acceptable salt thereof, is administered to a subject in need thereof in accordance with methods of the present disclosure for at least 8 weeks, at least 12 weeks, at least 14 weeks, at least 52 weeks, at least 2 years, at least 3 years, at least 4 years, at least 5 years or at least 10 years.

In some embodiments, methods of treating a disease or condition relating to aberrant function or activity of a T-type calcium channel, e.g., essential tremor, in a subject in need thereof, may result in a change, e.g., a decrease in TETRAS-mADL performance subscore score of the subject of 2 points or greater when the compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease in mADL11 performance subscore score of the subject of 2 points or greater when the compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least 0.5 points, at least 1 point, at least 2 points, at least 2.5 points, at least 3 points, at least 3.5 points, at least 4 points, at least 4.5 points, at least 5 points, at least 5.5 points, at least 6 points, at least 6.5 points, at least 7 points, at least 7.5 points, at least 8 points, at least 8.5 points, at least 9 points, at least 9.5 points or at least 10 points when the compound of formula (I) is administered at a dose of about 60 mg to about 100 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater when compound of formula (I) is administered at a dose of about 60 mg to about 100 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least 0.5 points, at least 1 point, at least 2 points, at least 2.5 points, at least 3 points, at least 3.5 points, at least 4 points, at least 4.5 points, at least 5 points, at least 5.5 points, at least 6 points, at least 6.5 points, at least 7 points, at least 7.5 points, at least 8 points, at least 8.5 points, at least 9 points, at least 9.5 points or at least 10 points when the compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater when compound of formula (I) is administered at a dose of about 60 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of at least 0.5 points, at least 1 point, at least 1.5 points, at least 2 points, at least 2.5 points, at least 3 points, at least 3.5 points, at least 4 points, at least 4.5 points, at least 5 points, at least 5.5 points, at least 6 points, at least 6.5 points, at least 7 points, at least 7.5 points, at least 8 points, at least 8.5 points, at least 9 points, at least 9.5 points, or at least 10 points when the compound of formula (I) is administered at a dose of about 100 mg once daily after an initial titration period.

In some embodiments, methods of treating a disease or condition relating to aberrant function or activity of a T-type calcium channel, e.g., essential tremor, in a subject in need thereof, may result in a change, e.g., a decrease, in TETRAS-mADL performance subscore score of the subject of 2 points or greater when compound of formula (I) is administered at a dose of about 100 mg once daily after an initial titration period.

In various aspects, the methods disclosed herein result in an EEG sigma frequency band reduction during NREM sleep in the subject, such as a NREM sigma band frequency reduction from a baseline of about 0.4 to 0.7, such as about 0.5 to about 0.6, or about 0.5. In certain embodiments, the methods disclosed herein result in an EEG gamma frequency band reduction during wake in an EO condition or an EC condition in the subject, such as a gamma frequency band reduction as compared to a baseline gamma frequency band of at least about 25%, such as, for example, about 50% reduction.

In certain embodiments, the methods disclosed herein result in an EEG sigma frequency reduction during NREM sleep and/or an EEG gamma frequency band reduction during an EO or an EC condition in the subject when the subject is administered a dosage of the compound of formula (I) or a pharmaceutically acceptable salt thereof resulting in a Cₘₐₓ of from about 30 ng/mL to about 470 ng/mL, such as a Cₘₐₓ ranging from about 30 to about 50 ng/mL, from about 80 to about 130 ng/mL, from about 130 to about 222 ng/mL, from about 180 to about 300 ng/mL, from about 230 to 380 ng/mL, or from about 280 to about 470 ng/mL. In certain embodiments, the methods disclosed herein result in an EEG sigma frequency reduction during NREM sleep and/or an EEG gamma frequency band reduction during an EO or an EC condition in the subject when the subject is administered a dosage of the compound of formula (I) or a pharmaceutically acceptable salt thereof resulting in an AUC₂₄ ranging from about 490 ng*h/mL to about 5800 ng*h/mL, such as an AUC₂₄ ranging from about 490 to 820 ng*h/mL, from about 1220 to 2030 ng*h/mL, from about 2000 to 3330 ng*h/mL, from about 2440 to 4070 ng*h/mL, from about 2820 to 4700 ng*h/mL, or from about 3480 to 5800 ng*h/mL.

In certain embodiments, the methods disclosed herein result in a NREM sigma frequency reduction in the subject when the subject is administered a dosage of the compound of formula (I) or a pharmaceutically acceptable salt thereof resulting in a Cₘₐₓ of about 5 ng/mL to about 470 ng/mL, such as a Cₘₐₓ of about 180 to about 300 ng/mL. In certain embodiments, the methods disclosed herein result in a NREM sigma frequency reduction in the subject when the subject is administered a dosage of the compound of formula (I) or a pharmaceutically acceptable salt thereof resulting in an average plasma concentration (Cₐᵥₑ) during the EEG recording (i.e., over a period of about 24 hours) of about 10 ng/mL to about 200 ng/mL, such as a Cₐᵥₑ of about 12 to about 150 ng/mL. In certain embodiments, the methods disclosed herein result in an EO or EC gamma frequency band reduction in the subject when the subject is administered a dosage of the compound of formula (I) or a pharmaceutically acceptable salt thereof resulting in a Cₘₐₓ of the compound of about, such as a Cₘₐₓ of about 280 to about 470 ng/mL. In certain embodiments, the methods disclosed herein result in an EO or EC gamma frequency band reduction in the subject when the subject is administered a dosage of the compound of formula (I) or a pharmaceutically acceptable salt thereof resulting in a plasma concentration of about 75 ng/ml to about 310 ng/mL, such as a plasma concentration of about 90 to about 190 ng/mL.

It certain aspects of all embodiments of titrated dosing schedules disclosed herein, it is possible to increase the maximum titrated dose, including, for example, increasing the maximum titrated dose above 120 mg in one or more additional titration steps, provided the subject is able to safely tolerate the higher dose.

In certain aspects of all of the embodiments of titrated dosing schedules disclosed herein, the maximum titrated dosage achieved is greater than 20 mg, greater than 40 mg, such as about 60 mg, about 80 mg, about 100 mg, about 120 mg, about 130 mg, about 140 mg, about 150 mg, about 160 mg, about 170 mg, about 180 mg, about 190 mg, about 200 mg, about 210 mg, or about 220 mg. In certain aspects of the disclosure, the maximum dosage for a subject is, for example, 40 mg, 60 mg, or 80 mg if the subject has achieved a desired therapeutic outcome.

In certain aspects of all of the embodiments of titrated dosing schedules disclosed herein, the maximum titrated dosage is reached in 42 days or less, such as 31 days or less, 28 days or less, 18 days or less, 10 days or less, or 7 days or less. In certain embodiments, the maximum titrated dosage is reached in about 10 to about 42 days, such as, for example, about 36-42 days, about 22-28 days, about 16-18 days, about 10-12 days, or about 7-10 days.

### Dosage Forms and Compositions

In some embodiments, the compound of formula (I) or a pharmaceutically acceptable salt thereof, e.g., the compound of formula (II) may be administered as a part of a dosage form or in a pharmaceutical composition.

In some embodiments, a composition that can be used in a method described herein may be a pharmaceutical composition comprising the compound of formula (I) or a pharmaceutically acceptable salt thereof, and an excipient that functions to modify the release rate of the compound of formula (I) or a pharmaceutically acceptable salt thereof. In some embodiments, the pharmaceutical composition may be a swellable core technology formulation.

In certain embodiments, a dosage form that can be used in a method described herein may be an oral dosage form comprising: the compound of formula (I) or a pharmaceutically acceptable salt thereof (e.g., the compound of formula (II)); and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit RS100)).

In other embodiments, a dosage form that can be used in a method described herein may be a dosage form or composition comprising the compound of formula (I) or a pharmaceutically acceptable salt thereof (e.g., the compound of formula (II)) and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit RS100)), for example, in an amount sufficient to modify the release rate of the compound of formula (I) or a pharmaceutically acceptable salt thereof (e.g., the compound of formula (II)) upon administration to the subject.

In some embodiments, the dosage form comprises from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight) of the compound of formula (I) or a pharmaceutically acceptable salt thereof (e.g., the compound of formula (II)). In some embodiments, the dosage form comprises about 30% by weight to about 40% by weight of the compound of formula (I) or a pharmaceutically acceptable salt thereof (e.g., the compound of formula (II)).

In some embodiments, the dosage form comprises from about 14% by weight to about 25% by weight of the compound of formula (I) or a pharmaceutically acceptable salt thereof (e.g., the compound of formula (II)). In some embodiments, the dosage form comprises from about 19% by weight to about 20% by weight of the compound of formula (I) or a pharmaceutically acceptable salt thereof (e.g., the compound of formula (II)). In some embodiments, the dosage form comprises from about 21% by weight to about 22% by weight of the compound of formula (I) or a pharmaceutically acceptable salt thereof (e.g., the compound of formula (II)). In some embodiments, the dosage form comprises from about 4% by weight to about 15% by weight of the compound of formula (I) or a pharmaceutically acceptable salt thereof (e.g., the compound of formula (II)). In some embodiments, the dosage form comprises from about 4% by weight to about 10% by weight of the compound of formula (I) or a pharmaceutically acceptable salt thereof (e.g., the compound of formula (II)). In some embodiments, the dosage form comprises form about 4% by weight to about 5% by weight of the compound of formula (I) or a pharmaceutically acceptable salt thereof (e.g., the compound of formula (II)). In some embodiments, the dosage form comprises from about 5% by weight to about 6% by weight of the compound of formula (I) or a pharmaceutically acceptable salt thereof (e.g., the compound of formula (II)). In some embodiments, the dosage form comprises from about 9% by weight to about 10% by weight of the compound of formula (I) or a pharmaceutically acceptable salt thereof (e.g., the compound of formula (II)).

In other embodiments, a dosage form that can be used in a method described herein may be a dosage form or composition comprising from about 1 mg to about 120 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg) of the compound of formula (I) or a pharmaceutically acceptable salt thereof (e.g., the compound of formula (II)) and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit RL100, Eudragit RS100)), for example, in an amount sufficient to modify the release rate of the compound of formula (I) or a pharmaceutically acceptable salt thereof (e.g., the compound of formula (II)) upon administration to the subject.

In other embodiments, the dosage form comprises from about 4 mg to about 6 mg (e.g., about 5 mg) of the compound of formula (I) or a pharmaceutically acceptable salt thereof (e.g., the compound of formula (II)). In certain embodiments, the dosage form comprises from about 15 mg to about 25 mg (e.g., about 20 mg) of the compound of formula (I) or a pharmaceutically acceptable salt thereof (e.g., the compound of formula (II)). In some embodiments, the dosage form comprises from about 5 mg to about 15 mg (e.g., about 10 mg) of the compound of formula (I) or a pharmaceutically acceptable salt thereof (e.g., the compound of formula (II)). In other embodiments, the dosage form comprises from about 25 mg to about 35 mg (e.g., about 30 mg) of the compound of formula (I) or a pharmaceutically acceptable salt thereof (e.g., the compound of formula (II)). In certain embodiments, the dosage form comprises from about 35 mg to about 45 mg (e.g., about 40 mg) of the compound of formula (I) or a pharmaceutically acceptable salt thereof (e.g., the compound of formula (II)). In certain embodiments, the dosage form comprises from about 45 mg to about 55 mg (e.g., about 50 mg) of the compound of formula (I) or a pharmaceutically acceptable salt thereof (e.g., the compound of formula (II)). In some embodiments, the dosage form comprises from about 55 mg to about 65 mg (e.g., about 60 mg) of the compound of formula (I) or a pharmaceutically acceptable salt thereof (e.g., the compound of formula (II)). In other embodiments, the dosage form comprises from about 65 mg to about 75 mg (e.g., about 70 mg) of the compound of formula (I) or a pharmaceutically acceptable salt thereof (e.g., the compound of formula (II)). In certain embodiments, the dosage form comprises from about 75 mg to about 85 mg (e.g., about 80 mg) of the compound of formula (I) or a pharmaceutically acceptable salt thereof (e.g., the compound of formula (II)). In certain embodiments, the dosage form comprises from about 85 mg to about 95 mg (e.g., about 90 mg) of the compound of formula (I) or a pharmaceutically acceptable salt thereof (e.g., the compound of formula (II)). In some embodiments, the dosage form comprises from about 95 mg to about 105 mg (e.g., about 100 mg) of the compound of formula (I) or a pharmaceutically acceptable salt thereof (e.g., the compound of formula (II)). In other embodiments, the dosage form comprises from about 105 mg to about 115 mg (e.g., about 110 mg) of the compound of formula (I) or a pharmaceutically acceptable salt thereof (e.g., the compound of formula (II)). In certain embodiments, the dosage form comprises from about 115 mg to about 125 mg (e.g., about 120 mg) of the compound of formula (I) or a pharmaceutically acceptable salt thereof (e.g., the compound of formula (II)).

In some embodiments, the dosage form comprises from about 55 mg to 65 mg of a modified-release polymer (e.g., an HPMC polymer). In some embodiments, the dosage form comprises from about 10% by weight to about 70% by weight of the modified-release polymer (e.g., an HPMC polymer). In some embodiments, the dosage form comprises from about 50% by weight to about 60% by weight of the modified-release polymer (e.g., an HPMC polymer).

In some embodiments, the dosage form further comprises a diluent. In some embodiments, the diluent comprises microcrystalline cellulose. In some embodiments, the dosage form comprises from about 15 mg to 40 mg (e.g., from about 15 mg to about 25 mg, from about 20 mg to about 25 mg, from about 25 mg to about 30 mg, from about 30 mg to about 40 mg) microcrystalline cellulose. In some embodiments, the dosage form comprises from about 15 mg to about 25 mg microcrystalline cellulose. In some embodiments, the dosage form comprises from about 30 mg to about 40 mg microcrystalline cellulose. In some embodiments, the dosage form comprises from about 15% to about 35% by weight (e.g., from about 15% to about 20%, from about 20% to about 25%, from about 25% to about 30%, from about 30% to about 35% by weight) microcrystalline cellulose.

In some embodiments, the dosage form further comprises a glidant. In some embodiments, the glidant comprises colloidal silicon dioxide. In some embodiments, the dosage form further comprises a lubricant. In some embodiments, the lubricant comprises magnesium stearate. In some embodiments, the dosage form further comprises a coating.

In some embodiments, about 80% of the compound of formula (I) or a pharmaceutically acceptable salt is released within 7 hours upon administration to a subject. In certain embodiments, about 80% of the compound of formula (I) or a pharmaceutically acceptable salt thereof is released in 7 hours using USP apparatus type-I, media containing 900 mL 0.1 M HCl, and a paddle speed of 100 rpm.

In some embodiments, the dosage form, upon administration to a subject, has a lower Cₘₐₓ value than a reference oral dosage form (e.g., a dosage form with any intended release rate profile e.g., modified release rate profile, a dosage form that does not have a modified release rate profile, e.g., an HPMC polymer). In some embodiments, the dosage form, upon administration to a subject, has a greater tₘₐₓ value than a reference oral dosage form (e.g., a dosage form with any intended release rate profile e.g., modified release rate profile, a dosage form that does not have a modified release rate profile, e.g., an HPMC polymer).

In some embodiments, the dosage form is administered to a patient once daily. In certain embodiments, the dosage form is administered to a patient twice daily. In some embodiments, the dosage form is a tablet. In other embodiments, the dosage form is a capsule. In certain embodiments, the dosage form is a suspension.

In some embodiments, a dosage form that can be used in a method described herein may be an oral dosage form (e.g., particulate) comprising: from about 15 mg to 25 mg of the compound of formula (I) or a pharmaceutically acceptable salt thereof (e.g., the compound of formula (II)); and from about 55 mg to 65 mg of an HPMC polymer.

In other embodiments, a dosage form that can be used in a method described herein may be an oral dosage form (e.g., particulate) comprising from about 14% by weight to about 25% by weight of the compound of formula (I) or a pharmaceutically acceptable salt thereof (e.g., the compound of formula (II)); and from about 53% to about 64% by weight of an HPMC polymer.

In certain embodiments, a dosage form that can be used in a method described herein may be an oral dosage form (e.g., particulate) comprising from about 3 mg to 8 mg of the compound of formula (I) or a pharmaceutically acceptable salt thereof (e.g., the compound of formula (II)); and from about 55 mg to 65 mg of an HPMC polymer.

In some embodiments, a dosage form that can be used in a method described herein may be an oral dosage form (e.g., particulate) comprising from about 3% by weight to about 8% by weight of the compound of formula (I) or a pharmaceutically acceptable salt thereof (e.g., the compound of formula (II)); and from about 53% to about 64% by weight of an HPMC polymer.

In other embodiments, a dosage form that can be used in a method described herein may be an oral (e.g., particulate) composition comprising the compound of formula (I) or a pharmaceutically acceptable salt thereof (e.g., the compound of formula (II)); and a modified-release polymer (e.g., a controlled-release polymer, e.g., an HPMC polymer as a hydrophilic matrix polymer).

In some embodiments, the composition comprises from about 0.9% by weight to about 40% by weight of the compound of formula (I) or a pharmaceutically acceptable salt thereof (e.g., the compound of formula (II)). In some embodiments, the composition comprises from about 14% by weight to about 25% by weight of the compound of formula (I) or a pharmaceutically acceptable salt thereof (e.g., the compound of formula (II)). In some embodiments, the composition comprises about 19% by weight to about 20% by weight of the compound of formula (I) or a pharmaceutically acceptable salt thereof (e.g., the compound of formula (II)). In some embodiments, the composition comprises about 21% by weight to about 22% by weight of the compound of formula (I) or a pharmaceutically acceptable salt thereof (e.g., the compound of formula (II)). In some embodiments, the composition comprises from about 4% by weight to about 15% by weight of the compound of formula (I) or a pharmaceutically acceptable salt thereof (e.g., the compound of formula (II)). In some embodiments, the composition comprises from about 4% by weight to about 10% by weight of the compound of formula (I) or a pharmaceutically acceptable salt thereof (e.g., the compound of formula (II)). In some embodiments, the composition comprises about 4% by weight to about 5% by weight of the compound of formula (I) or a pharmaceutically acceptable salt thereof (e.g., the compound of formula (II)). In some embodiments, the composition comprises about 5% by weight to about 6% by weight of the compound of formula (I) or a pharmaceutically acceptable salt thereof (e.g., the compound of formula (II)). In some embodiments, the composition comprises about 9% by weight to about 10% by weight of the compound of formula (I) or a pharmaceutically acceptable salt thereof (e.g., the compound of formula (II)).

In some embodiments, the composition comprises from about 1 mg to about 120 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg) of the compound of formula (I) or a pharmaceutically acceptable salt thereof (e.g., the compound of formula (II)). In certain embodiments, the composition comprises from about 4 mg to about 6 mg (e.g., about 5 mg) of the compound of formula (I) or a pharmaceutically acceptable salt thereof (e.g., the compound of formula (II)). In other embodiments, the composition comprises from about 15 mg to about 25 mg (e.g., about 20 mg) of the compound of formula (I) or a pharmaceutically acceptable salt thereof (e.g., the compound of formula (II)). In certain embodiments, the composition comprises from about 25 mg to about 35 mg (e.g., about 30 mg) of the compound of formula (I) or a pharmaceutically acceptable salt thereof (e.g., the compound of formula (II)). In other embodiments, the composition comprises from about 35 mg to about 45 mg (e.g., about 40 mg) of the compound of formula (I) or a pharmaceutically acceptable salt thereof (e.g., the compound of formula (II)). In certain embodiments, the composition comprises from about 45 mg to about 55 mg (e.g., about 50 mg) of the compound of formula (I) or a pharmaceutically acceptable salt thereof (e.g., the compound of formula (II)). In some embodiments, the composition comprises from about 55 mg to about 65 mg (e.g., about 60 mg) of the compound of formula (I) or a pharmaceutically acceptable salt thereof (e.g., the compound of formula (II)). In other embodiments, the composition comprises from about 65 mg to about 75 mg (e.g., about 70 mg) of the compound of formula (I) or a pharmaceutically acceptable salt thereof (e.g., the compound of formula (II)). In certain embodiments, the composition comprises from about 75 mg to about 85 mg (e.g., about 80 mg) of the compound of formula (I) or a pharmaceutically acceptable salt thereof (e.g., the compound of formula (II)). In certain embodiments, the composition comprises from about 85 mg to about 95 mg (e.g., about 90 mg) of the compound of formula (I) or a pharmaceutically acceptable salt thereof (e.g., the compound of formula (II)). In some embodiments, the composition comprises from about 95 mg to about 105 mg (e.g., about 100 mg) of the compound of formula (I) or a pharmaceutically acceptable salt thereof (e.g., the compound of formula (II)). In other embodiments, the composition comprises from about 105 mg to about 115 mg (e.g., about 110 mg) of the compound of formula (I) or a pharmaceutically acceptable salt thereof (e.g., the compound of formula (II)). In certain embodiments, the composition comprises from about 115 mg to about 125 mg (e.g., about 120 mg) of the compound of formula (I) or a pharmaceutically acceptable salt thereof (e.g., the compound of formula (II)).

In some embodiments, the composition comprises a diluent. In some embodiments, the diluent comprises microcrystalline cellulose. In other embodiments, the composition comprises from about 15 mg to 40 mg (e.g., from about 15 mg to about 25 mg, from about 20 mg to about 25 mg, from about 25 mg to about 30 mg, from about 30 mg to about 40 mg), microcrystalline cellulose. In some embodiments, the composition comprises from about 15% to about 35% by weight (e.g., from about 15% to about 20%, from about 20% to about 25%, from 25% to about 30%, from 30% to about 35% by weight) microcrystalline cellulose.

In some embodiments, the composition comprises from about 15 mg to about 25 mg microcrystalline cellulose. In some embodiments, the composition comprises from about 30 mg to about 40 mg microcrystalline cellulose. In some embodiments, the composition further comprises a glidant. In some embodiments, the glidant comprises colloidal silicon dioxide. In some embodiments, the composition further comprises a lubricant. In some embodiments, the lubricant comprises magnesium stearate. In some embodiments, the composition further comprises a coating. In some embodiments, the compound of formula (I) or (II), including, for example, the compound of Form C or Form B, is stable within the formulation at about 25° C. at 60% relative humidity for at least 24 months. In some embodiments, the compound is stable at about 25° C. at 60% relative humidity for at least 36 months. In some embodiments, the compound is stable at about 25° C. at 60% relative humidity for at least 48 months. In other embodiments, the compound is stable at about 25° C. at 60% relative humidity for at least 60 months. In some embodiments, the compound is stable at about 40° C. at 75% relative humidity for at least 6 months.

In other embodiments of the oral dosage forms or compositions described herein, the compound of formula (I) or a pharmaceutically acceptable salt thereof (e.g., the compound of formula (II)) is a crystalline form. In certain embodiments, the crystalline form is a crystalline form as described herein, such as crystalline Form C or crystalline Form B. In other embodiments, the crystalline form is crystalline Form D. In certain embodiments, the crystalline form is a crystalline form previously or contemporaneously described, such as crystalline Pattern B, crystalline Pattern C or crystalline Pattern D. Crystalline Pattern B and crystalline Pattern C are described, e.g., in WO 2021/007487, the entire contents of which are hereby incorporated herein by reference. Crystalline Pattern D is characterized by an x-ray powder diffraction (XRPD) pattern comprising at least one peak at the diffraction angle (° 2θ) selected from the group consisting of a peak at approximately 12.0°; a peak at approximately 15.6°; a peak at approximately 16.7°; a peak at approximately 19.8°; a peak at approximately 21.2°; a peak at approximately 24.10; a peak at approximately 25.2°; a peak at approximately 27.3°; and a peak at approximately 30.2°.

### Crystalline Form of the Compound of Formula (II)

A crystalline form of the compound of formula (II) used in the method as described herein may exhibit an X-ray powder diffraction (XRPD) pattern comprising at least one peak selected from peaks at the following diffraction angles (2θ): 26.6±0.2, 16.2±0.2, 17.4±0.2, 22.6±0.2, 11.5±0.2, 23.9±0.2, 18.3±0.2, 19.2±0.2, 18.5±0.2 or 20.0±0.2. In certain embodiments, a crystalline form of the compound of formula (II) disclosed herein may exhibit an XRPD pattern comprising peaks at the following diffraction angles (2θ): 16.2±0.2, 17.4±0.2, and 26.6±0.2.

In some embodiments, the crystalline form exhibits an XRPD pattern comprising peaks at the following diffraction angles (2θ): 11.5±0.2, 16.2±0.2, 17.4±0.2, 22.6±0.2, and 26.6±0.2. In other embodiments, the crystalline form exhibits an XRPD pattern comprising peaks at the following diffraction angles (2θ): 11.5±0.2, 16.2±0.2, 17.4±0.2, 18.3±0.2, 18.5±0.2, 19.2±0.2, 20.0±0.2, 22.6±0.2, 23.9±0.2, and 26.6±0.2. In some embodiments, the XRPD pattern was obtained using Cu Kα radiation. In certain embodiments, the crystalline form has a melting point onset as determined by differential scanning calorimetry at about 226.6° C.

A crystalline form of the compound of formula (II) used in the method as described herein may exhibit an XRPD pattern comprising at least one peak selected from peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2 or 21.2±0.2. In certain embodiments, a crystalline form of the compound of formula (II) disclosed herein may exhibit an XRPD pattern comprising peaks at the following diffraction angles (2θ): 21.9±0.2, 18.5±0.2, and 17.8±0.2.

In some embodiments, the crystalline form exhibits an XRPD pattern comprising peaks at the following diffraction angles (2θ): 21.9±0.2, 18.5±0.2, 17.8±0.2, 10.2±0.2, and 20.5±0.2. In other embodiments, the crystalline form exhibits an XRPD pattern comprising peaks at the following diffraction angles (2θ): 21.9±0.2, 18.5±0.2, 17.8±0.2, 10.2±0.2, 20.5±0.2, 25.2±0.2, 16.9±0.2, 24.2±0.2, 28.6±0.2, and 21.2±0.2. In some embodiments, the powder XRPD was obtained using Cu Kα radiation. In certain embodiments, the crystalline form has a melting point onset as determined by differential scanning calorimetry at about 97.9° C., 131.6° C., 223.7° C., 83.8° C., 128.9° C., 168.9° C., or 224.4° C.

### Immediate-Release Formulations

In some embodiments, a dosage form or composition that can be used in a method described herein may be a dosage form or composition comprising the compound of formula (I) or a pharmaceutically acceptable salt thereof (e.g., the compound of formula (II)), where the compound of formula (I) or a pharmaceutically acceptable salt thereof (e.g., the compound of formula (II)) is released immediately upon an administration to the subject.

In other embodiments, a dosage form that can be used in a method described herein may be an oral capsule for immediate release comprising from about 15 mg to about 20 mg of the compound of formula (I) or a pharmaceutically acceptable salt thereof (e.g., the compound of formula (II)); from about 75 mg to 85 mg diluent; from about 2 mg to 10 mg binder; from about 1% to about 5% disintegrant; and from about 0.1 mg to 5 mg lubricant.

### Administrations

In some embodiments, the dosage form is administered to the subject more than once a day (e.g., twice a day, three times a day, or four times a day).

In some embodiments, the dosage form is administered to the subject once a day (e.g., one 20 mg tablet once a day, two 20 mg tablets (or one 40 mg tablet) once a day, or three 20 mg tablets (or one 60 mg tablet) once a day). In some embodiments, the dosage form is administered to the subject twice a day (e.g., one 10 mg tablet twice a day, one 20 mg tablet twice a day, two 20 mg tablets twice a day, three 20 mg tablets twice a day). In some embodiments, the dosage form is administered to the subject every other day, twice a week, or once a week. In certain embodiments, a dose of about 1 mg to 60 mg, such as 20 mg to 40 mg, of the compound of formula (I) or a pharmaceutically acceptable salt thereof (e.g., the compound of formula (II)) is administered to the subject daily. In other embodiments, a dose of about 15 mg to 25 mg of the compound of formula (I) or a pharmaceutically acceptable salt thereof (e.g., the compound of formula (II)) is administered to the subject daily. In certain embodiments, a dose of about 30 mg to 40 mg of the compound of formula (I) or a pharmaceutically acceptable salt thereof (e.g., the compound of formula (II)) is administered to the subject daily.

In some embodiments, the dosage form, upon administration to the subject, has a reduced Cₘₐₓ value in comparison to a reference oral dosage form (e.g., a dosage form with any intended release rate profile e.g., modified release rate profile, a dosage form that does not have a modified release rate profile, e.g., an HPMC polymer). In some embodiments, the dosage form, upon administration to the subject, has a greater tₘₐₓ value than a reference oral dosage form (e.g., a dosage form with any intended release rate profile e.g., modified release rate profile, a dosage form that does not have a modified release rate profile, e.g., an HPMC polymer).

### Tremor

The methods described herein can be used to treat tremor, for example a dosage or composition disclosed herein can be used to treat cerebellar tremor or intention tremor, dystonic tremor, essential tremor, orthostatic tremor, parkinsonian tremor, physiological tremor, or rubral tremor. Tremor includes hereditary, degenerative, and idiopathic disorders such as Wilson's disease (hereditary), Parkinson's disease (degenerative), and essential tremor (idiopathic); metabolic diseases; peripheral neuropathies (associated with Charcot-Marie-Tooth, Roussy-Levy, diabetes mellitus, complex regional pain syndrome); toxins (nicotine, mercury, lead, carbon monoxide, manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be a neuropathic tremor, and can be classified into physiologic tremor, enhanced physiologic tremor, essential tremor syndromes (including classical essential tremor), primary orthostatic tremor, task- and position-specific tremor, dystonic tremor, parkinsonian tremor, cerebellar tremor, Holmes' tremor (i.e., rubral tremor), palatal tremor, toxic or drug-induced tremor, and psychogenic tremor. The tremor may be familial tremor.

Tremor is an involuntary, rhythmic, oscillation of one or more body parts (e.g., hands, arms, eyes, face, head, vocal folds, trunk, and/or legs).

Cerebellar tremor or intention tremor is a slow, broad tremor of the extremities that occurs after a purposeful movement. Cerebellar tremor is caused by lesions in or damage to the cerebellum or pathways resulting from, e.g., tumor, stroke or other focal lesion disease (e.g., multiple sclerosis) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and nonprogressive in some, or may be slowly progressive, starting on one side of the body but typically affecting both sides. Essential tremor may also be moderate or severe. The hands are most often affected, but the head, voice, tongue, legs, and trunk may also be involved. Tremor frequency may decrease as a person ages, but, alternatively, severity may increase. Heightened emotion, stress, fever, other illness, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor. In an embodiment, the tremor is essential tremor. In various embodiments, the essential tremor is with or without intention tremor. In an embodiment, the essential tremor is without intention tremor.

The efficacy of the compound or composition described herein for treating essential tremor can be measured by methods known in the art, such as the methods described in the following references: Ferreira, J. J. et al., “MDS Evidence-Based Review of Treatments for Essential Tremor,” Mov. Disord. 2019 July; 34(7):950-958; Elble, R. et al., “Task Force Report: Scales for Screening and Evaluating Tremor,” Mov. Disord. 2013 November; 28(13):1793-800; Deuschl G. et al., “Treatment of patients with essential tremor,” Lancet Neurol. 2011; 10:148-61; and Reich S. G. et al., “Essential Tremor,” Med. Clin. N. Am. 2019; 103:351-356. The disclosures of the references are herein incorporated in their entirety.

In some embodiments, the methods described herein result in at least 25% reduction in the upper limb tremor score, wherein the tremor score may be converted to amplitude, as compared to a baseline. For example, in certain embodiments, the methods described herein result in about 40% mean reduction in tremor amplitude as measured by The Essential Tremor Rating Assessment Scale (TETRAS) upper limb score, described, for example, in Elble, R. J., “The Essential Tremor Rating Assessment Scale,” J. Neurol. Neuromed. 2016; 1(4):34-38. In some embodiments, the methods described herein result in at least 25% reduction in TETRAS performance score as compared to the baseline. In some embodiments, the methods described herein result in at least 35% average reduction in symptom severity as compared to the baseline, as measured by TETRAS performance score.

### Combination Therapy

In accordance with methods of the present disclosure, the compound of formula (I), or a pharmaceutically acceptable salt thereof, may be administered in combination with one or more other agent or therapy. For example, the compound of formula (I), or a pharmaceutically acceptable salt thereof, may be administered to a subject in combination with one or more tremor medications. Exemplary tremor medications which may be administered in combination with compound of formula (I), or a pharmaceutically acceptable salt thereof, in accordance with methods of the present disclosure include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide. In one embodiment, the tremor medication is propranolol. In another embodiment, the tremor medication is primidone.

In an embodiment, tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### Examples

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

### Example 1. Phase 3 Trial to Evaluate the Efficacy of the Compound of Formula (1) in the Treatment of Adults with Essential Tremor

### Overview

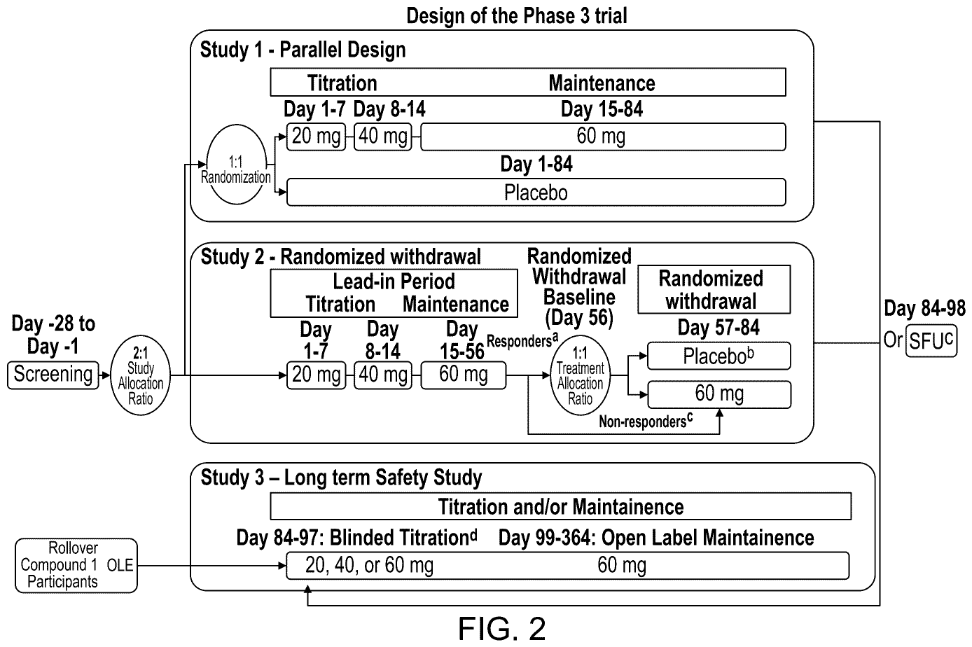

This Phase 3 study is an ongoing, three-part, randomized, double-blind, placebo-controlled, fixed-dose (after titration) trial that compares the efficacy and safety of Compound 1 with placebo in participants aged 18 to 80 years who have a diagnosis of ET. The design of the study is illustrated in FIG. 2 and includes Study 1, Study 2 and Study 3. The primary objective of Study 1 parallel design (PD) is to evaluate the change from Baseline in mADL11 score for Compound 1 compared to placebo after 12 weeks (84 days) of treatment. The primary objective of Study 2 randomized withdrawal (RW) is to evaluate the maintenance of response by measuring the proportion of participants that maintain response following RW. Other efficacy assessments in Studies 1 and 2 include Clinical Global Impression-Severity (CGI-S), Patient Global Impression-Severity (PGI-S), Patient Global Impression-Change (PGI-C), and Archimedes spiral drawing. The primary objective of Study 3 long-term safety study (LTSS) is to assess the long-term safety of up to approximately 1 year of Compound 1 treatment. The safety and tolerability of Compound 1 will be assessed throughout all 3 studies.

This is a decentralized Phase 3 clinical trial, utilizing a combination of in-home and telehealth visits, and consisting of 3 simultaneous studies that include a randomized, DB, placebo-controlled PD study, a DB RW study, and an LTSS study. The studies in this trial are designed to evaluate the efficacy and/or safety of Compound 1 in participants aged 18 to 80 years who have a diagnosis of ET and have had symptoms for at least 3 years. Study 1 PD and Study 2 RW participants will be informed of all 3 studies and must agree during informed consent that, if deemed eligible, they will be assigned to either Study 1 PD or Study 2 RW and rollover into Study 3 LTSS. A specific informed consent form (ICF) will be used for Phase 2 OLE participants that rollover into Study 3 LTSS.
In Study 1 PD, participants will receive 12 weeks (84 days) of either Compound 1 or matching placebo in a DB fashion.
In Study 2 RW, participants will receive 8 weeks (56 days) of Compound 1 in a DB fashion before entering a 4-week DB RW period, where responders will undergo RW and non-responders will continue Compound 1 treatment. Responder participants in the RW period who then meet the predefined relapse criteria will rollover into Study 3 LTSS with blinded dose titration as described in Table 6.
In Study 3 LTSS participants will receive up to a total of approximately 1 year of Compound 1, which includes prior study dosing.

Compound 1 will be titrated to 60 mg over a 2-week period (Table 8). After the baseline visit, participants will administer either Compound 1 or placebo every morning (QAM) at home prior to their scheduled home health or telehealth visits, as applicable. Dose adjustments will not be allowed. Participants who complete Studies 1 PD or Study 2 RW (complete all assessments through Day 84), meet the predefined relapse criteria in Study 2 RW, or complete a Phase 2 EOT visit may rollover into Study 3 LTSS, with Day 84 assessments carried forward from their respective studies.

The duration of Studies 1 PD and 2 RW can be as long as 18 weeks, including the Screening Period (Table 4) and the Treatment and Safety Follow-up (SFU) Periods (Table 5 and Table 6). The duration of the LTSS will be up to approximately 1 year (Table 7).

FIG. 2 is a schematic showing the design of the Phase 3 trial.

TABLE 4
Pivotal Studies: Screening Period

| Study Period | Description |
|---|---|
| Screening | 4 weeks with weekly visits as described below: |
| Period | Home Health Visit: Day −21 |
| | Telehealth Visits: Day −28, Day −21, Day −14, and Day −7 |

TABLE 5
Study 1 PD: Intervention and Safety-Follow Up Periods

| Study Period | Description |
|---|---|
| Baseline Visit | Day 1: Home Health Visit and Telehealth Visit |
| Treatment | 12-week duration |
| Period | Titration Period: 2 weeks |
| | Week 1 (Days 1 through 7) 20 mg QAM or |
| | placebo |
| | Week 2 (Days 8 through 14) 40 mg QAM or |
| | placebo |
| | Maintenance Period: 10 weeks |
| | Week 3 to Week 12 (Days 15 through 84) 60 |
| | mg QAM or placebo |
| Safety | 2 weeks (14 days) following the last dose of |
| Follow-up | study drug for those who either complete the |
| Period | study and choose not to participate in Study 3 |
| | LTSS or discontinue treatment early for any |
| | reason. |

Abbreviations: LTSS = long-term safety study; QAM = every morning.

TABLE 6
Study 2 RW: Intervention and Safety Follow-Up Periods

| Study Period | Description |
|---|---|
| Baseline Visit | Day 1: Home Health Visit and Telehealth Visit |
| DB Intervention | 8-week duration |
| Period: RW | Titration Period: 2 weeks |
| Lead-in Period | Week 1 (Days 1 through 7) 20 mg QAM |
| | Week 2 (Days 8 through 14) 40 mg QAM |
| | Maintenance Period: 6 weeks |
| | Week 3 to Week 8 (Days 15 through 56) 60 mg |
| | QAM |
| DB Intervention | 4-week duration |
| Period: RW | Randomized Compound 1 Responder |
| Period | Cohort: Randomized 1:1 to either continue |
| | Compound 1 treatment at their current dose (60 |
| | mg) or transition to placebo. |
| | Relapse Group: Transfer to Study 3 LTSS and |
| | either maintain Compound 1 or begin 2-week |
| | titration from placebo to 60 mg. |
| | Nonrandomized Cohort: Remain on Compound 1 |
| | 60 mg |
| Safety | 2 weeks (14 days) following the last dose of study |
| Follow-up | drug for those who either complete the study and |
| Period | choose not to participate in Study 3 LTSS or |
| | discontinue treatment early for any reason. |

Abbreviations: DB = double blind; LTSS = long-term safety study; QAM = every morning; RW = randomized withdrawal.

TABLE 7
Study 3 LTSS: Intervention and Safety Follow-Up Periods

| Study Period | Description |
|---|---|
| LTSS | Day 84: Telehealth Visit for Phase 2 OLE participants, |
| Baseline | after informed consent and investigator review of |
| | most recent OLE safety assessments. |
| | Study 1 and 2 participants that rollover into LTSS will |
| | enter at Day 84.^{a} |
| Intervention | Up to approximately 1 year in duration, with DB |
| Period | administration of Compound 1 through Week 14. |
| | Blinded Titration Period^{b}: 2 weeks |
| | Days 84 through 90: 20 mg QAM (or maintain 60 mg) |
| | Days 91 through 97: 40 mg QAM (or maintain 60 mg) |
| | Maintenance Period: 50 weeks |
| | Day 99 up to approximately 1 year: 60 mg QAM |
| Safety | 2 weeks (14 days) following the last dose of study drug |
| Follow-up | for those who either complete the study and choose |
| Period | not to continue in Study 3 LTSS or discontinue |
| | treatment early for any reason. |

Abbreviations: DB = double blind; EOT = end of treatment; LTSS = long-term safety study; OLE = open label extension; PD = parallel design; QAM = every morning; RW = randomized withdrawal.
^{a}For rollover participants, Day 84/EOT assessments should be carried over from Study 1 PD or Study 2 RW as the LTSS baseline.
^{b}A blinded titration period will occur during the first two weeks of Study LTSS for participants rolling over from Studies 1 and 2. During this blinded titration period, participants will either titrate from their placebo dose to 60 mg over a 2-week period or maintain their 60 mg dose.

Eligible participants will be randomized to receive oral Compound 1 or placebo QAM. Fixed titration regimens will be used, as described in Table 8.

Dose adjustments will not be allowed.

TABLE 8
Intervention Period Dosing Sequence by Treatment Group

| | Intervention Period | | | |
|---|---|---|---|---|
| Study | Titration | | Maintenance | |
| Study 1 PD | Week 1 | Week 2 | Week 3 to Week 12 | |
| | (7 days) | (7 days) | (70 days) | |
| Study 2 RW | | | Lead-in | Randomized |
| | | | Period | Withdrawal |
| | | | Week 3 to | Week 9 to |
| | | | Week 8 | Week 12 ^{a} |
| | | | (42 days) | (28 days) |
| Study 3 LTSS | Week 1 | Week 2 | Maintenance | |
| | (7 days) ^{b} | (7 days) ^{b} | Week 3^{c} up to 1 Year | |
| Treatment | Dose | | | |
| Compound 1 | 20 mg | 40 mg | 60 mg | |
| Study 1 PD | Week 1 | Week 2 | Week 3 to Week 12 | |
| | (7 days) | (7 days) | (70 days) | |
| Study 2 RW | | | Lead-in | Randomized |
| | | | Period | Withdrawal |
| | | | Week 3 to | Week 9 to |
| | | | Week 8 | Week 12 ^{a} |
| | | | (42 days) | (28 days) |

| Placebo | Matching Placebo |
|---|---|
| Study | Duration |
| Study 1 PD | 84 Days |
| Study 2 RW | 84 Days |
| Study 3 LTSS | Up to approximately 1 Year |

LTSS = long term safety study; OLE = open label extension; PD = parallel design; RW = randomized withdrawal.
^{a} In Study 2 RW, after 6 weeks of maintenance dosing, participants that meet the responder criteria will receive either 4 weeks of placebo or ulixacaltamide. The remainder of participants will remain on ulixacaltamide.
^{b} Only for participants who are not currently on ulixacaltamide (i.e., are on placebo in Study 1 PD or Study 2 RW prior to entering Study 3 LTSS). For participants rolling over from Study 1 PD and Study 2 RW, dosing over the first 2 weeks of the LTSS study will remain blinded.
^{c}Participants entering Study 3 LTSS after completing Study 1 PD or Study 2 RW will remain on a 60 mg dose or begin titration of ulixacaltamide to 60 mg as defined in the schedule above. Participants entering Study 3 LTSS from PRAX-944-222 OLE will remain on 60 mg or titrate over a two-week period to 60 mg as follows: if their current dose is greater than 60 mg they will reduce their dose directly to 60 mg and if lower than 60 mg they will titrate based on the titration schedule described above, with a dose increase every 7 days based on their current dose (i.e. 20 or 40 mg).

The objectives and endpoints for Study 1 PD are described below (Table 9).

TABLE 9
Objectives and Endpoints for Study 1 PD

| | |
|---|---|
| Objectives | Endpoints |

Primary
Efficacy

| | |
|---|---|
| To evaluate the efficacy of | Change from Baseline to Day 84 on the |
| Compound 1 compared with | mADL11 |
| placebo in participants with ET | |

Secondary
Efficacy

| | |
|---|---|
| To further evaluate the efficacy | Proportion of participants responding |
| of Compound 1 compared with | to ulixacaltamide, as defined by |
| placebo in participants with ET | change in mADL11 score after 12 |
| over time | weeks (84 days) of treatment |
| | Change from Baseline to Day 84 in: |
| | TETRAS-ADL |
| | PGI-C |
| | CGI-S |
| | PGI-S |
| | Proportion of participants responding |
| | to ulixacaltamide, as defined by |
| | change in mADL11 score after 14, 28, |
| | 56, and 70 days of treatment |
| | Change from Baseline to Day 14, Day |
| | 28, Day 56, and Day 70 in: |
| | mADL11 |
| | TETRAS-ADL |
| | PGI-C |
| | CGI-S |
| | PGI-S |
| | Dominant Hand Archimedes Spiral |
| | (Day 1, Day 56, Day 84) |

Safety

| | |
|---|---|
| To evaluate the safety of | Incidence and severity of AEs, |
| Compound 1 compared to | including discontinuation of study |
| placebo in participants with ET | drug due to AEs |
| | Changes in vital sign measurements |
| | Changes in clinical laboratory results |
| | Changes in ECG parameters |
| | Incidence of C-SSRS measured suicidal |
| | ideation or behavior |
| | Changes in BDI-II and BAI |

Exploratory
Clinical Pharmacology

| | |
|---|---|
| To determine plasma | Plasma Compound 1 concentrations |
| concentrations of Compound 1 | |
| in participants with ET | |

AE = adverse events; BAI = Beck Anxiety Index; BDI-II = Beck Depression Inventory - Second Edition; CGI-S = Clinical Global Impressions-Severity; C-SSRS = Columbia-Suicide Severity Rating Scale; ECG = electrocardiogram; ET = essential tremor; mADL11 = modified TETRAS-ADL items 1 to 11 with modified score; PGI-C = Patient Global Impression of Change; PGI-S = Patient Global Impression of Severity; TETRAS-ADL = The Essential Tremor Rating Assessment Scale - Activities of Daily Living.

The objectives and endpoints for Study 2 RW are described below (Table 10).

TABLE 10
Objectives and Endpoints for Study 2 RW

| | |
|---|---|
| Objectives | Endpoints |

Primary
Efficacy

| | |
|---|---|
| To evaluate Compound 1 | The proportion of participants that |
| efficacy maintenance of | maintain response, as defined by change |
| response in the RW portion | in mADL11 score, following RW |
| of the study | |

Secondary
Efficacy

| | |
|---|---|
| To further evaluate the | Change from RW Baseline (Day 56) to |
| efficacy of continued | Day 63, Day 70, Day 77, and Day 84 on |
| Compound 1 over time | the following: |
| | mADL11 |
| | TETRAS-ADL |
| | PGI-C |
| | CGI-S |
| | PGI-S |
| | Dominant hand Archimedes Spiral |
| | (Day 1, Day 56, Day 84) |

Safety

| | |
|---|---|
| To evaluate the safety | Incidence and severity of AEs, |
| of Compound 1 in | including discontinuation of study drug |
| participants with ET | due to AEs |
| | Changes in vital sign measurements |
| | Changes in clinical laboratory results |
| | Changes in ECG parameters |
| | Incidence of C-SSRS measured suicidal |
| | ideation or behavior |
| | Changes in BDI-II and BAI |

Exploratory
Clinical Pharmacology

| | |
|---|---|
| To determine plasma | Plasma Compound 1 concentrations |
| concentrations of | |
| Compound 1 in | |
| participants with ET | |

AE = adverse events; BAI = Beck Anxiety Index; BDI-II = Beck Depression Inventory - Second Edition; CGI-S = Clinical Global Impression of Severity; CSSRS = Columbia-Suicide Severity Rating Scale; ECG = electrocardiogram; ET = essential tremor; mADL11 = modified TETRAS-ADL items 1 to 11 with modified score; PGI-C = Patient Global Impression of Change; PGI-S = Patient Global Impression of Severity; RW = randomized withdrawal; TETRAS-ADL = The Essential Tremor Rating Assessment Scale-Activities of Daily Living.

The objectives and endpoints for Study 3 LTSS are described below (Table 11).

TABLE 11
Objectives and Endpoints for Study 3 LTSS

| | |
|---|---|
| Objective | Endpoints |

Primary

| | |
|---|---|
| To evaluate the long-term | Incidence and severity of AEs |
| safety of Compound 1 in | Changes in vital sign measurements |
| participants with ET | Changes in clinical laboratory results |
| | Changes in ECG parameters |
| | Incidence of C-SSRS measured |
| | suicidal ideation or behavior |

AE = adverse events; C-SSRS = Columbia-Suicide Severity Rating Scale; ECG = electrocardiogram; ET = essential tremor.

### Background and Clinical Trial Rationale

Compound 1 is a novel small molecule being developed for the treatment of essential tremor (ET) that is a high-affinity, state-dependent, selective inhibitor of T-type calcium ion (Ca₂₊) channels. Comprised of 3 isoforms (CaV3.1, CaV3.2, and CaV3.3), T-type Ca²⁺ channels are expressed widely throughout the brain, especially in the cerebello-thalamo-cortical (CTC) circuit. Aberrant bursts in the CTC circuit occur at the same frequency as upper limb tremor in ET.

ET is the most common adult movement disorder, affecting up to 2% of the United States (US) population with an estimated true prevalence of approximately 7 million Americans. ET is characterized by a 6 to 12 Hz postural and kinetic tremor (i.e., tremor during voluntary movement) in the upper limbs. The most characteristic clinical feature is kinetic tremor of the arms and hands, but tremor may also occur in the head and voice as the disease progresses and, less commonly, in the face, legs, and trunk. Emerging evidence supports the understanding that ET represents a heterogeneous syndrome and diagnosis requires further differentiation based on medical history and neurological exam.

ET has a significant impact on several dimensions of daily functioning in patients, especially writing, eating, and drinking. Functional disability was reported in over 70% of patients with ET, and was associated with comorbid anxiety and depression.

Despite being one of the most common movement disorders, ET often remains undertreated or untreated, in part because of the limited number of available treatments. Propranolol and primidone are the medications used most frequently to treat ET. Propranolol is the only FDA-approved orally administered treatment indicated for the treatment of ET. Propranolol was originally developed and approved for hypertension in 1967 and was approved for the treatment of ET in 1986 on the basis of a 2-week clinical trial that enrolled only 9 patients and evaluated a limited dose range of 40 mg to 80 mg three-times daily. As scientific understanding of ET has matured and clinical experience with propranolol has been gained, propranolol has emerged as an inadequate, poorly tolerated treatment, leaving a severe unmet medical need for patients with ET. Propranolol in the treatment of ET exhibits limited efficacy with side effects (e.g., bradycardia) that often lead to discontinuation. The American Academy of Neurology 2011 ET treatment guideline states that not all patients improve on or tolerate primidone and propranolol. The most recent data indicate that for patients who are receiving pharmacotherapy, over 40% discontinue all pharmacological options after 2 years of treatment. Thus, while there is at least 1 approved therapy, the currently available medications for ET fail to meet the needs of most patients.

The results of the Phase 2 trials with ulixacaltamide, which are described in Examples 1 and 2 of WO 2024/182677, the entire contents of which are hereby incorporated herein by reference, support the efficacy and safety of ulixacaltamide in patients with ET. The Phase 2 trials demonstrated reduced modified ADL (mADL) scores (i.e., reduced tremor symptoms) at daily doses ranging from 20 to 120 mg for up to 56 days. In the double-blind (DB) portion of the Phase 2 study, the rescored TETRAS-ADL items 1-11 (mADL11) score decreased more in participants randomized to Compound 1 compared with placebo after 56 days of dosing, with least square mean (LSM standard error [SE]) scores of (2.69 [0.81]) compared with placebo (−0.87 [0.95]). The LSM (95% confidence interval [CI]) treatment difference was −1.82 (95% CI: −3.565, −0.066; with a nominal p value of <0.05). In the Phase 2 extension period, 20 participants completed a blinded 6-week crossover period. Participants were randomized in blinded fashion to either 3 weeks of Compound 1 60 mg or placebo. Rescue treatment (Compound 1) was offered to participants if their mADL11 worsened by a preset threshold. During the crossover period, maintenance of response was observed in 17 of 20 participants receiving Compound 1 compared to 11 of 21 participants receiving placebo, with a Chi-squared p-value of 0.0249 and 0.0388 when adjusted for propranolol use, family history and intention tremor (IT). None of the Compound 1-treated participants and 9 of 21 placebo-treated participants qualified for and received rescue treatment within the 3-week period. No new safety signal was identified during this portion of the study.

Across the clinical development program, Compound 1 has been generally well tolerated following up to 129-days of daily administration at doses ranging from 5 to 120 mg, demonstrating a favorable safety profile following a titration schedule. No deaths or serious adverse events (SAEs) considered by the sponsor as being related to study drug have been reported following administration of Compound 1. Likewise, no suicide attempts or pregnancies have been reported following administration of Compound 1.

This Phase 3 study is a three-part, randomized, DB, placebo-controlled, fixed-dose (after titration) trial that will compare the efficacy and safety of Compound 1 with placebo in participants aged 18 to 80 years who have a diagnosis of ET. The primary objective of Study 1 parallel design (PD) is to evaluate the change from Baseline in mADL11 score for Compound 1 compared to placebo after 12 weeks (84 days) of treatment. The primary objective of Study 2 randomized withdrawal (RW) is to evaluate the maintenance of response by measuring the proportion of participants that maintain response following RW. Other efficacy assessments in Studies 1 and 2 include Clinical Global Impressions-Severity (CGI-S), Patient Global Impression-Severity (PGI-S), Patient Global Impression-Change (PGI-C) and Archimedes spiral drawing. The primary objective of Study 3 long-term safety study (LTSS) is to assess the long-term safety of up to approximately 1 year of Compound 1 treatment. The safety and tolerability of ulixacaltamide will be assessed throughout all 3 studies.

### Overall Design

This trial is a decentralized, Phase 3, multi-study, clinical trial evaluating the safety and efficacy of Compound 1 in ET. The trial includes 2 separate simultaneous pivotal phase 3 studies with all participants undergoing one screening process. This simultaneous study design promotes a more homogeneous study population and ensures blinded study assignment and treatment arm allocation across Study 1 PD and Study 2 RW, an objective that could not be met if Study 2 RW were conducted in isolation.

The trial consists of 3 simultaneous, decentralized, studies including 2 pivotal DB placebo-controlled studies and an LTSS in participants with ET as follows: Study 1 PD: A 12-week PD, randomized, DB, placebo-controlled study to evaluate the change from Baseline in mADL11 score for Compound 1 compared to placebo after 12 weeks (84 days) of treatment.

Study 2 RW: A 12-week RW, DB, placebo-controlled study to evaluate the efficacy and maintenance of response and durability of effect in participants who respond to Compound 1 and safety.

Study 3 LTSS: An LTSS to evaluate the long-term safety of Compound 1 for up to approximately 1 year. The first 2 weeks of the LTSS will include a 2-week blinded titration period for participants who rollover from Study 1 PD and Study 2 RW to maintain the blind for these studies. The remainder of the study will be open label. Participants who are enrolled in the Phase 2 open label extension (OLE) can rollover to Study 3 LTSS and will either begin titration over a 2-week period to 60 mg or continue taking the 60 mg dose.

Both pivotal studies include a Screening Period and all 3 studies have Intervention and Safety Follow-up Periods. There are two types of study visits: home health visits and telehealth visits. Home health visits will be staffed by a trained research nurse traveling to the participant's home. The investigator and/or research staff will participate in home health visits remotely with audiovisual communication. The telehealth visit will be a remote audiovisual visit with the investigator and/or research staff. Investigator will supervise all visits. In addition to home health and telehealth visits, study drug will be directly mailed to participants.

### Screening and Baseline Periods

Key screening assessments include medical history, demographics, prior and concomitant medications, physical and neurologic examination, drug/alcohol screen, pregnancy screen, clinical laboratory evaluations, electrocardiogram (ECG), vital signs, Columbia-Suicide Severity Rating Scale (C-SSRS), and assessments of ET severity using The Essential Tremor Rating Assessment Scale-Activities of Daily Living (TETRAS-ADL).

The Screening Period for Compound 1 naïve participants will be 28 days in duration. After all screening assessments are completed and a participant is deemed eligible for the study, randomization will occur between Day −7 and Day −3 to allow time for assigned study drug delivery to the participant's home.

Participants will complete the TETRAS-ADL assessment 3 times during Screening (on Day −28, Day −14, and Day −7).

Phase 2 OLE participants will not be required to complete this screening process, but the investigator must review the most recent safety assessments for these participants approximately 1 week before their first LTSS study visit. If safety issues are identified, the investigator will review with the sponsor to determine if participant is eligible to continue Compound 1 in the LTSS. Throughout the Intervention Periods in Studies 1 PD, 2 RW, or 3 LTSS, participants who choose or are required to stop study drug dosing must have an early discontinuation (ED) visit scheduled as soon as possible. If participants choose not to participate in the Study 3 LTSS after completion of Study 1 PD or Study 2 RW, they must enter the 14-day Safety Follow-up Period. Participants who prematurely discontinue from Study 1 PD or Study 2 RW will not be eligible to participate in the LTSS.

Participants will self-administer either ulixacaltamide or placebo every morning (QAM) at home prior to their scheduled home health or telehealth visits, as applicable. Key efficacy assessments will be performed at select visits based on study assignment and include TETRAS-ADL, PGI-C, PGI-S, CGI-S, and Archimedes spiral drawing. Drug concentration samples (on Day 56 and Day 70) will be drawn at the same time as clinical laboratory assessments, following the correct collection order.

### Study 1 PD

All participants assigned to Study 1 PD who continue to meet all clinical trial entry criteria on Day 1 will receive DB treatment with Compound 1 or placebo from Day 1 through Day 84, using a fixed-dose regimen (following titration).

On Baseline (Day 1) participants will receive 1 tablet of study drug during the home health visit and then self-administer QAM from Day 2 to Day 84. Assessments for CGI-S, PGI-S, and Archimedes Spiral will be performed in that order before dosing.

For participants randomized to Compound 1, the titration period will consist of a 20 mg QAM dose during Week 1 (Days 1 to 7) and a 40 mg QAM dose during Week 2 (Days 8 to 14). On Day 15, these participants will enter the maintenance period, receiving a 60 mg QAM dose through the end of the Intervention Period (Day 84). No dosage adjustments will be allowed.

Participants who complete Study 1 PD can rollover into the ongoing Study 3 LTSS with Day 84 assessments carried forth from Study 1 PD.

### Study 2 RW

All participants assigned to Study 2 RW, who continue to meet all clinical trial entry criteria on Day 1, will enter the RW Lead-in Period.

### RW Lead-in Period

During the RW Lead-in Period participants will be administered Compound 1 during the Home Health Visit on Day 1 and self-administered from Day 2 through Day 56. These participants will be blinded to whether they are participating in this study or Study 1 PD and will therefore be administered Compound 1 in a DB fashion.

On Day 1 (Baseline) participants will complete CGI-S, PGI-S, and Archimedes Spiral assessments in that order before dosing.

All participants will undergo a titration period that will consist of a 20 mg QAM dose during Week 1 (Days 1 to 7) and a 40 mg QAM dose during Week 2 (Days 8 to 14). On Day 15, these participants will enter the maintenance period, receiving a 60 mg QAM dose through the end of the Intervention Period (Day 56). No dosage adjustments will be allowed.

### RW Period

Based on pre-defined selection criteria, a subset of participants who are responders to Compound 1 will be randomized 1:1 to either continue Compound 1 or receive placebo in a DB fashion. No Compound 1 taper is required prior to starting placebo. The participants who do not meet these criteria will remain on Compound 1 at 60 mg throughout the remainder of the study. Study visits will be conducted weekly, throughout the duration of the RW period as outlined in the SoA.

Participants who complete Study 2 RW can rollover into the ongoing Study 3 LTSS with Day 84 assessments carried forth from Study 2 PD.

Participants who meet the criteria of worsening tremor (according to the predefined relapse criterion) during the RW, will rollover to Study 3 LTSS. The final assessments conducted in Study 2 RW will carry forth to Study 3 LTSS Day 84.

### Study 3 LTSS

Participants on placebo in Studies 1 PD or 2 RW will undergo a DB titration period that will consist of a 20 mg QAM dose during the first week (Days 84 to 90) and a 40 mg QAM dose during the second week (Days 91 to 97) (Table 9). Participants who were receiving 60 mg Compound 1 in a pivotal study when rolling over to the LTSS will remain on the 60 mg dose throughout the titration period in a blinded fashion. On Day 15, all participants will enter the open-label maintenance period, receiving a 60 mg QAM dose through the remainder of the trial. Participants in the Phase 2 OLE (described in Example 2 of WO 2024/182677) will rollover to Study 3 LTSS at the 60 mg dose or titrate over a 2-week period to 60 mg if appropriate based on their current dose. No dosage adjustments will be allowed in Study 3 LTSS.

### Scientific Rationale for Clinical Trial Design

This Protocol includes 2 simultaneous pivotal DB studies designed to further assess the favorable efficacy and safety profiles of Compound 1 demonstrated throughout the Phase 2 program. A multi-study protocol offers multiple benefits over a traditional single trial protocol. Shared trial infrastructure allows more efficiency in enrollment, operations, data collection and management as well as overall resource utilization across the 3 studies. Furthermore, simultaneous study enrollment ensures a more homogeneous patient population across the two pivotal Phase 3 trials (Study 1 PD and Study 2 RW). By initiating Study 1 PD and Study 2 RW simultaneously under one protocol, administration of Compound 1 treatment in the RW Lead-in Period of Study 2 RW will be blinded, resulting in a more accurate determination of Compound 1 responders. Finally, all Study 1 PD and Study 2 RW completers can directly enter the LTSS, allowing seamless transition from a participant perspective and continuous safety data collection from a sponsor perspective.

Study 1 PD and Study 2 RW will be conducted using 2 different study designs. Study 1 PD is similar to the Phase 2 trial described in Example 1 of WO 2024/182677, but extends the total treatment period to 12 weeks to allow assessment of chronic treatment. The Study 2 RW design was chosen for multiple reasons. First, durability of effect will be further confirmed in the RW arm designed to assess a subpopulation defined as ulixacaltamide responders. The RW design is expected to be useful since ET is known to be heterogeneous; and given this heterogeneity (i.e., unique patient characteristics, genotype/etiology or phenotype), it is likely that there are subpopulations of ET participants that will respond more favorably to treatment than other subpopulations. The RW design identifies this subpopulation, their response to treatment, and will therefore lead to a better understanding of the responder population. In addition, participants will be assessed weekly during the RW period, and those who meet the predefined relapse criterion will rollover into Study 3 LTSS. This limits the duration of placebo for those that are true responders.

The Intervention Periods in Studies 1 PD and 2 RW are up to 12 weeks in duration, including the 2-week titration. A 2 week titration period is considered appropriate based on the tolerability observed in Phase 2. Additionally, a 10-week maintenance treatment period at the 60 mg dose is considered sufficient to establish the durability of the effect of chronic dosing. This is supported by evidence of a treatment effect as early as 2 weeks in the Phase 2 trials. In Phase 2, the LSM mADL11 score change from baseline showed a treatment effect relative to placebo of 1.44 on Day 28, with sustained effect throughout the remainder of the study (Day 56). A favorable treatment effect of 1.03 points in mADL11 relative to placebo was observed as early as the first assessment point (Day 14) in participants without intention tremor (IT).

In the Phase 2 OLE study (described in Example 2 of WO 2024/182677), the requirement for rescue treatment in the placebo group was observed within the 3-week crossover period suggesting a rapid washout out of drug effect and further supporting the 4-week RW period chosen for Study 2 RW.

The presence of IT is of special interest because it is a distinct type of action tremor observed in some individuals with ET that can be associated with other unique neurologic conditions. IT is characterized by a high amplitude, low frequency coarse tremor that worsens with movement toward a target as opposed to the action or postural tremor otherwise observed in ET in which tremor frequency and amplitude is more consistent throughout the trajectory of movement. Because IT can be associated with a diagnosis other than ET, participants with IT will be assessed during screening to determine if there is a potential cerebellar etiology different than that of ET. Only participants determined by the Eligibility Review Committee (ERC) central reviewer to have IT associated with ET (ET with IT) and not an alternative etiology for IT will be enrolled in the study. Functional, physiologic and anatomical data suggests that ET with IT may represent a unique clinical manifestation that can create a potentially confounding effect in terms of clinical study outcome measures based on functional activities. Therefore, this subpopulation could confound the measurement and interpretation of the primary outcome measure, ADL performance. Based on this, participants with IT will be enrolled only if IT is determined to be associated with ET and not an alternative etiology. Participants assigned to Studies 1 PD or 2 RW will be stratified for the presence of ET with IT. Participants determined to have ET with IT will be included in Study 1 PD and Study 2 RW sensitivity analyses.

The primary endpoints for Study 1PD and Study 2 RW are based on mADL11 (TETRAS-ADL items 1-11 with transformed score 0-3). In the Phase 2 trial described in Example 1 of WO 2024/182677, the primary endpoint was mADL (TETRAS-ADL items 1-11+ performance subscale [PS] items 6 and 7 with transformed score 0-3). While mADL score was reduced in the Phase 2 studies, careful analysis of the data from the Phase 2 trial described in Example 1 of WO 2024/182677 revealed that the PS items (Archimedes spirals and cursive handwriting) and ADL item 12 (social impact) did not have sufficient sensitivity, reliability, specificity, and/or clinical meaningfulness to assess ET severity. Thus, in alignment with feedback from the FDA, the mADL11 score was chosen as the primary efficacy endpoint for Study 1 PD and as a secondary endpoint for Study 2 RW. Dominant hand Archimedes spiral will be analyzed as a secondary endpoint.

This is a decentralized clinical trial. The decreased burden of a decentralized trial would enhance involvement of participants without access to research clinics, participation of older participants that are limited by transportation (and other issues, e.g., mobility, etc.), and younger participants who are potentially still working and need a more convenient clinical trial schedule. This will improve trial participant engagement, recruitment, enrollment, and retention not only of a meaningfully diverse clinical population but also can increase investigator diversity which is important for clinical trials in the US. Furthermore, home-based collection of efficacy assessments is ideal since study assessments are functionally relevant ADLs that focus on participants daily experience and disability associated with ET. The Phase 2 crossover study shows that a decentralized trial can be effectively and safely performed for participants with moderate to severe ET, and the TETRAS-ADL can be assessed via telehealth with consistency in mADL11 observed over multiple consecutive timepoints. Given this, these assessments are feasible to collect, and bias can be reduced through collection by a decentralized investigator. Finally, a decentralized trial is appropriate given the well-tolerated and well-characterized safety profile of Compound 1 as noted in the Phase 2 study as described in Example 1 of WO 2024/182677. Compound 1 has stable characteristics that allow direct-to-patient shipping, simple drug administration, and simple safety monitoring appropriate for a decentralized trial.

### End of Clinical Trial Definition

A participant is considered to have completed the Studies 1 PD and 2 RW when they either rollover to Study 3 LTSS or complete a SFU or ED visit. The end of Study 3 LTSS will occur when participants have completed the last planned assessment, as shown in SoA. The trial may be terminated at any time by the sponsor.

### Justification for Dose

Ulixacaltamide, when administered as the modified release (MR) formulation, in the context of titration, has demonstrated a favorable safety profile in healthy participants and patients with ET throughout the Phase 2 development program at doses ranging from 5 to 120 mg for up to 129 days.

In the Phase 2 trial described in Example 1 of WO 2024/182677, participants were randomized to placebo or 60 or 100 mg of Compound 1. When evaluating the change from baseline versus placebo on the mADL11 endpoint, the magnitude of benefit was largely similar between participants in the 60 mg or 100 mg Compound 1 groups. There was no additional benefit observed by increasing the dose to 100 mg and no significant difference in the safety profile between the 60 mg and 100 mg groups. Thus, the benefit:risk ratio for the 60 mg dose is ideal without the need to evaluate additional maintenance doses lower than 60 mg.

The effect of a high-fat, high-calorie meal on the pharmacokinetics of Compound 1 was evaluated in healthy participants who had previously received Compound 1 under fasted conditions in a Phase 1 trial. The effect of a high-fat, high-calorie meal on the PK of Compound 1 was evaluated at the 120 mg dose level after healthy adult participants had titrated to reach steady-state exposure. Following administration of 120 mg dose in the fed state or fasted state, the maximum plasma drug concentration (Cₘₐₓ) and AUC_{τ} values were similar. Based on this analysis, Compound 1 may be administered without regard to food.

### Study Drug Administration

Compound 1 will be supplied as 20 mg, 40 mg, and 60 mg modified-release tablets. Matching placebo will also be supplied. Study drug is administered orally QAM with or without food and will be provided to participants in pre-packaged bottles. Participants receiving ulixacaltamide will receive 1 tablet of study drug per day. Participants in the placebo group will receive one tablet of matching placebo per day. For each home health/telehealth visit, the dose must be taken from the previously dispensed bottle. The interactive response technology (IRT) system can dispense replacement bottles, if necessary.

Eligible participants will be randomized to receive oral Compound 1 or placebo daily. Fixed titration regimens will be used for the 3 studies beginning with 20 mg for 7 days, then 40 mg for 7 days followed by 60 mg maintenance dose (note that participants dosed with placebo in Study 1 PD and Study 2 RW will require blinded titration upon rollover into Study 3 LTSS). Dose adjustments will not be allowed.

### Efficacy Assessments

All TETRAS-ADL assessments throughout the study will be conducted by a trained and qualified investigator. The Baseline CGI-S, PGI-S, and Archimedes Spiral assessments on Day 1 (Baseline) must be conducted pre-dose.

### The Essential Tremor Rating Assessment Scale

The TETRAS was developed by the Tremor Research Group (TRG; www.tremorresearchgroup.org) to quantify ET severity and its impact on ADLs. The full scale has 2 sections, the Performance and ADL subscales. This study will collect only the TETRAS-ADL.

### TETRAS Activities of Daily Living

The TETRAS-ADL subscale is a 12-item assessment of typical daily activities that are impacted by tremor. Activities are assessed in the following functional domains: speaking, feeding, drinking, personal hygene, dressing, pouring, carrying, using keys, writing, working, overall disability and social activity. The impact to each function is rated on a 5-point Likert scale from 0 to 4. The full ADL subscale score is calculated as the sum of all 12 items and ranges from 0 to 48.

### TETRAS Modified Activities of Daily Living 11

The mADL11 score is a composite sum of items 1 to 11 (omission of item 12 social impact score) of the TETRAS-ADL subscale. Scoring of each item is transformed to a 4-point scale from 0 to 3 with total score range from 0 to 33. The TETRAS ADL will be collected and the mADL11 will be used for data analysis purposes.

### Clinical Global Impression

The Clinical Global Impressions (CGI) scales are commonly used measures of symptom severity and response in clinical trials (Guy 1976). The CGI-S scale will be used in this clinical trial.

### Clinical Global Impression-Severity

The CGI-S assesses the clinician's impression of the participant's current illness state. The clinician should use his or her total clinical experience with this patient population and rate the current severity of the participant's ET on a 7-point scale from 1 (normal, not at all ill) to 7 (among the most extremely ill patients).

### Patient Global Impression

### Patient Global Impression-Change

The PGI-C assesses the participant's improvement (or worsening). The participant is required to assess his or her condition relative to Baseline (Pre-dose on Day 1) on a 7-point scale from 1 (very much improved) to 7 (very much worse). In all cases, the assessment should be made independent of whether the participant believes the improvement/worsening is drug-related or not.

### Patient Global Impression-Severity

The PGI-S assesses the participant's impression of his or her current illness state. The participant is required to assess his or her condition on a 7-point scale from 1 (Not present) to 7 (extremely severe).

### Archimedes Spiral

Participants will draw Archimedes Spirals with their dominant hand on an unlined secured standard size (letter) paper, which includes preformed spiral drawings. A specific pen (to standardize across all drawings) will be distributed for use. Participants will be instructed to draw a spiral without crossing lines in the preformed spiral template using their dominant hand, without practice, and without leaning on the dominant forearm, wrist or hand. Descriptive analyses will be used to summarize the findings.

### Preliminary Results of Eligibility Screening

Preliminary eligibility to participate in the Phase 3 study was assessed via an online pre-screener capturing demographics, symptoms, and other clinical features. Assessment of qualifiers was based on, though not limited to, the following: receiving an ET diagnosis; bilateral arm/hand tremor; symptoms >3 years; sufficient self-reported action tremor severity with higher likelihood of meeting inclusion criteria. The data below are presented for 75,000 survey responders.

Responders were of median age 71 (18-101) years. Of those with an ET diagnosis, 58% indicated being diagnosed/treated by a neurologist. Most (70%) were diagnosed in the last 10 years. Between 71% and 87% of responders reported difficulty completing daily tasks, e.g., pouring, drinking from a glass, picking up/reaching for small items, using a smartphone. Conversely, between 12% and 29% of responders indicated no problem completing tasks. Most responders had not received ultrasound/surgery, Cala Trio, or botulinum toxin intervention. Over 85% of responders reported willingness to allow home study visits, with 91% willing to use a mobile app and video calls if deemed eligible to participate.

The findings to date from this largest ET survey to date show that patients seeking participation in a clinical trial report ET as a high-burden, inadequately managed disorder impacting several activities of daily living. Findings also suggest a degree of normalization of ET impact (likely necessitating ongoing patient and physician education) and emphasize the urgent need for innovation in developing effective, well-tolerated treatments.

### Update: Characteristics of Adult Essential Tremor Patients Seeking Participation in the Phase 3 Trial

Patients can participate from home in the Phase 3 trial. The screening and participation involves: 1) a patient completes online pre-screener; 2) patient call with study coordinator; 3) telehealth visit with a study physician; 4) at home nurse visit; and 5) at home nurse and telehealth visits.

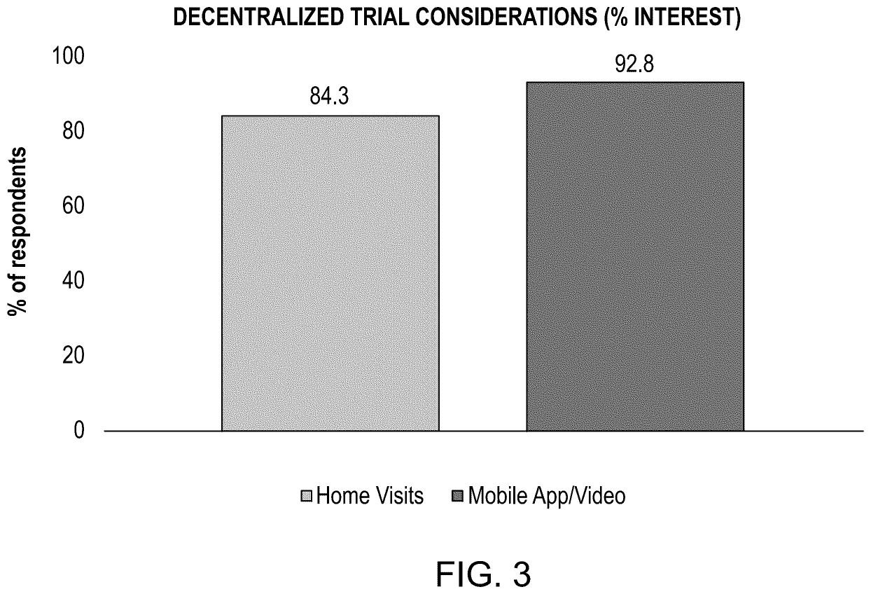

FIG. 3 is a bar graph showing percent of responders willing to allow home visits and willing to use a mobile app and video calls. The results shown in FIG. 3 indicate that ~85% of pre-screener responders report willingness to allow home study visits, with >90% willing to use a mobile app and video calls if deemed eligible to participate.

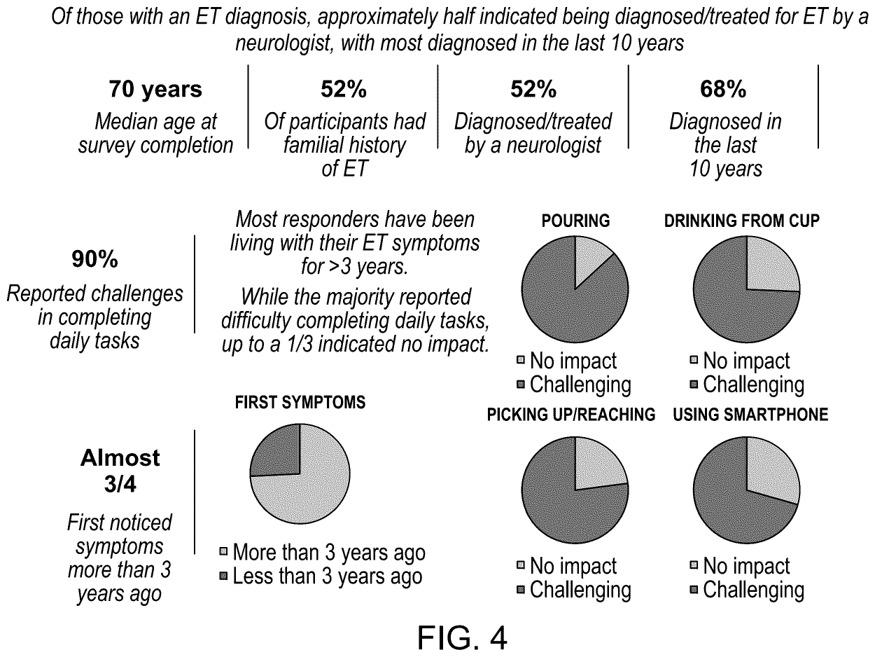

FIG. 4 is a series of pie charts demonstrating that the essential tremor is underdiagnosed, with widespread impact on daily living. The responders were of a median age of 70 years old. Of the responders, 52% had familial history of essential tremor which was diagnosed and treated by a neurologist, and 68% of those diagnosed were diagnosed in the last 10 years. Most responders have been living with their essential tremor symptoms for >3 years, and almost ¾ of responders first noticed their symptoms more than 3 years ago. While the majority reported difficulty completing daily tasks, up to ⅓ of responders indicated no impact.

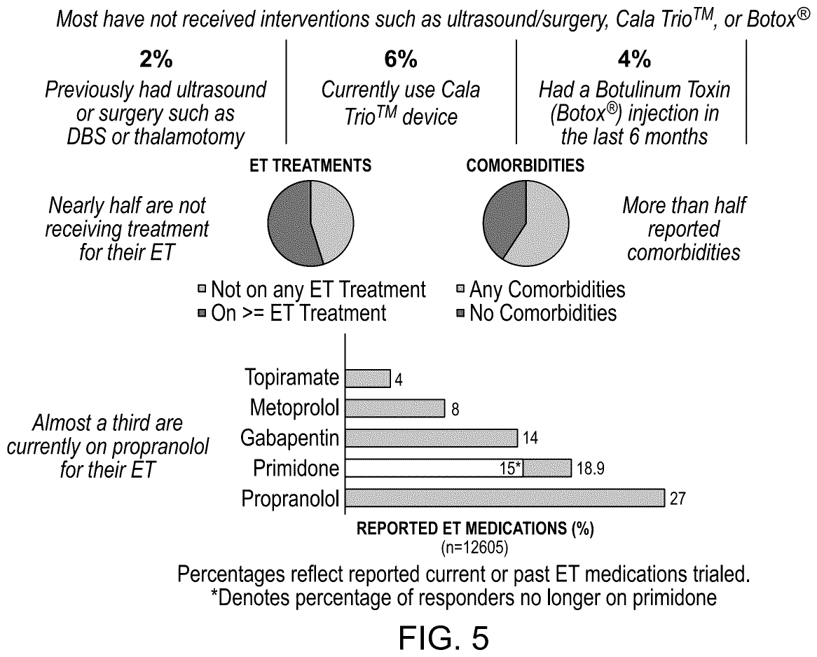

FIG. 5 is a series of pie charts and bar graphs demonstrating essential tremor treatments and comorbidity burden. Most responders have not received interventions, such as ultrasound/surgery, Cala Trio™, or Botox®. Nearly half of responders are not receiving treatment for their essential tremor, and more than half of responders reported comorbidities. Almost a third of responders are currently on propranolol for their essential tremor.

### Conclusions

This Phase 3 trial comprised two simultaneous Phase 3 studies including a 12-week, parallel design study and 12-week randomized withdrawal study for stable responders. Data are presented for over 175,000 pre-screener responders, of which over 27,000 referrals met pre-qualifying eligibility criteria for the Phase 3 trial.

The findings show that patients seeking participation in a clinical trial report essential tremor as a high-burden, inadequately managed disorder impacting several activities of daily living. Findings also suggest a degree of normalization of essential tremor impact (likely necessitating ongoing patient and physician education) and emphasize the urgent need for innovation in developing effective, well-tolerated treatments.

### Topline Results

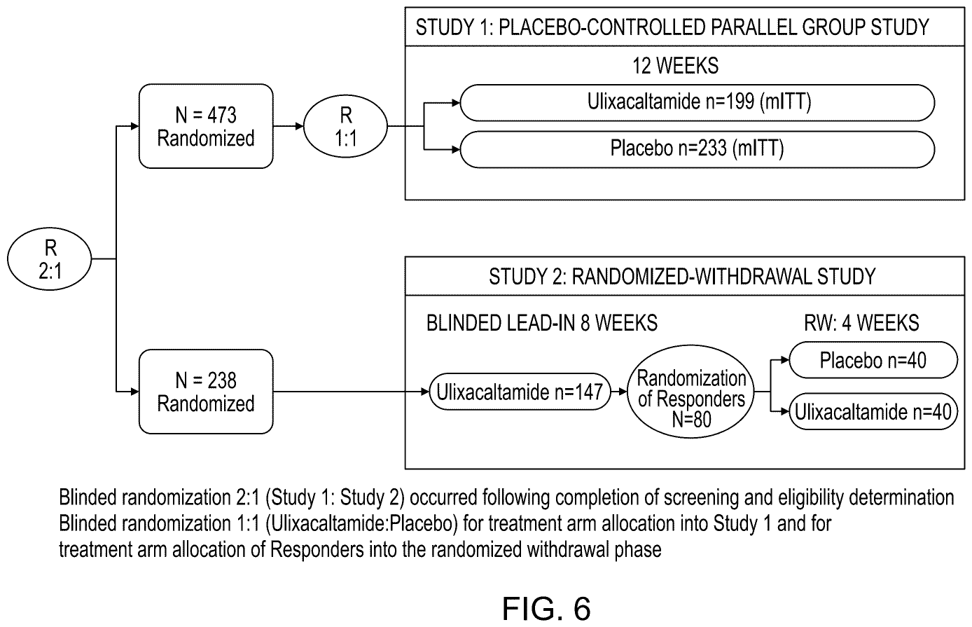

FIG. 6 is a schematic of the phase 3 trial described in this example which consisted of Study 1 (placebo-controlled parallel group study) and Study 2 (randomized-withdrawal study).

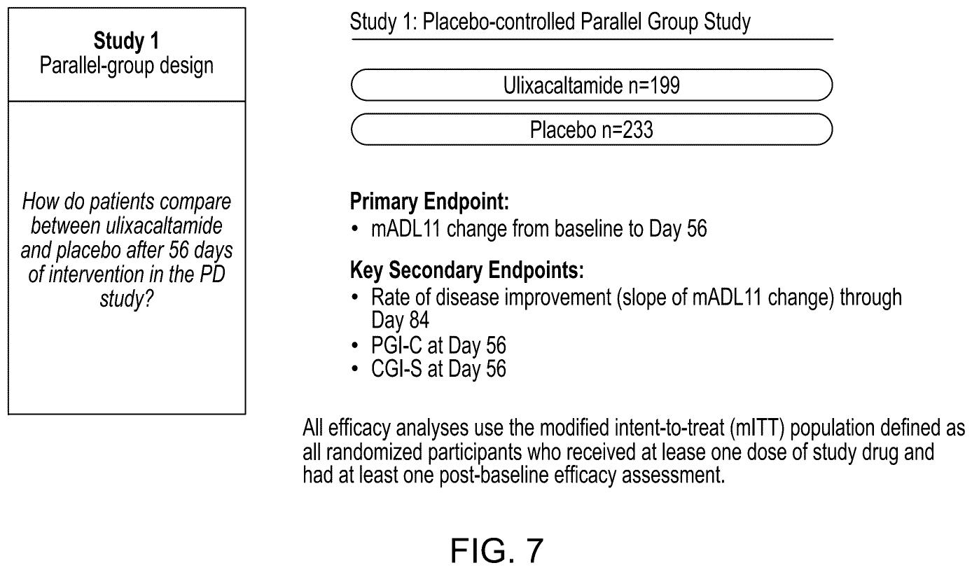

FIG. 7 is a schematic illustrating the analysis of the Study 1 data. The analysis illustrated a comparison of patients between ulixacaltamide and placebo after 56 days of intervention in the parallel design (PD) study.

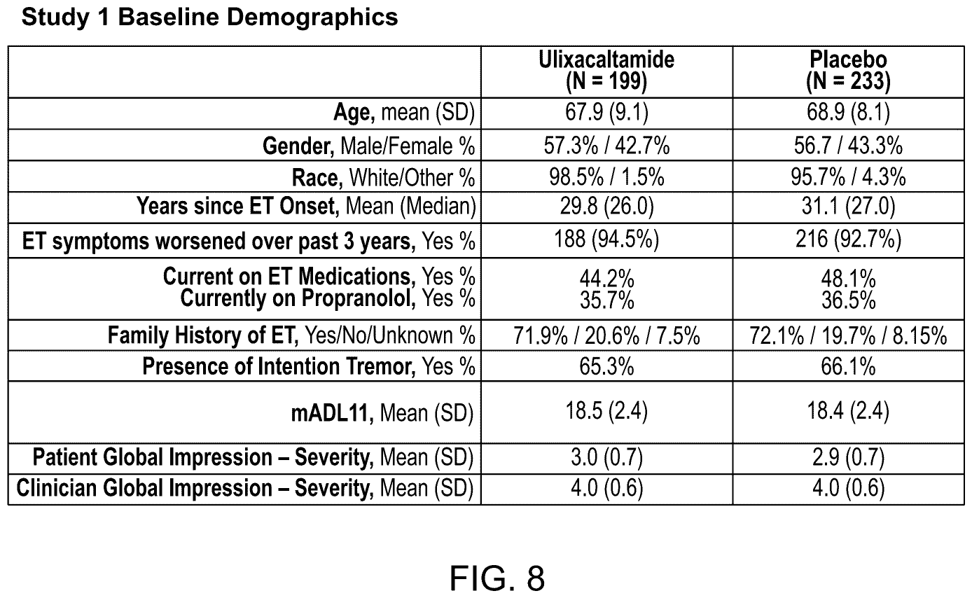

FIG. 8 is a table describing baseline demographics of the participants of Study 1.

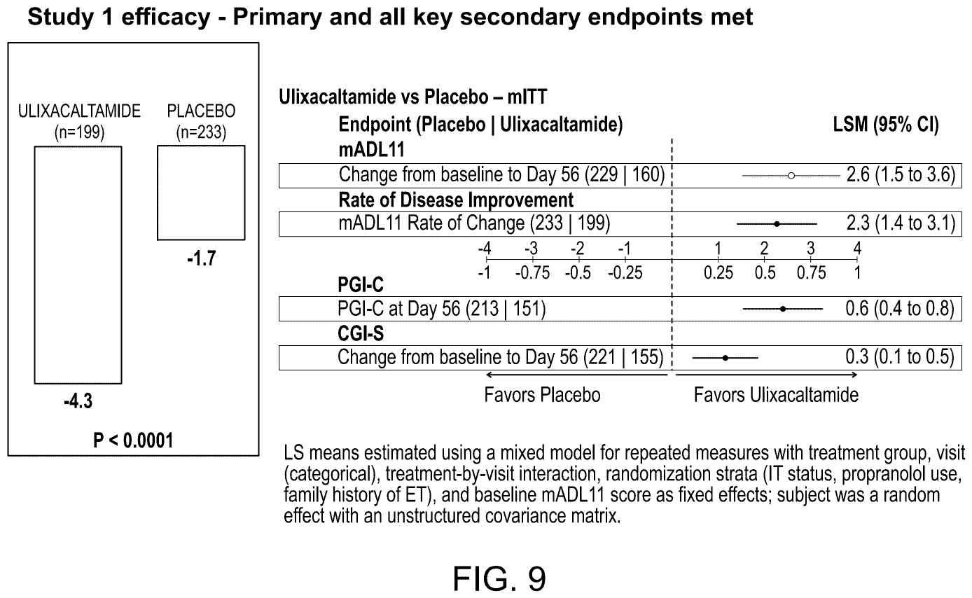

FIG. 9 is a series of graphs illustrating efficacy of ulixacaltamide in Study 1. The bar graph on the left in FIG. 9 shows that participants that were administered ulixacaltamide for 56 days in Study 1 (n=199) experienced a decrease in mADL11 score of 4.3 points, while the subjects administered placebo (n=233) experienced a decrease in mADL11 score of 1.7 points. The graph on the right in FIG. 9 shows the results of least square means (LSM) analysis as it relates to changes in primary endpoint (mADL11 change from baseline) and secondary endpoints (rate of disease improvement through Day 84; PGI-C at day 56 and CGI-S at day 56). The LSM analysis demonstrates that the results favor ulixacaltamide.

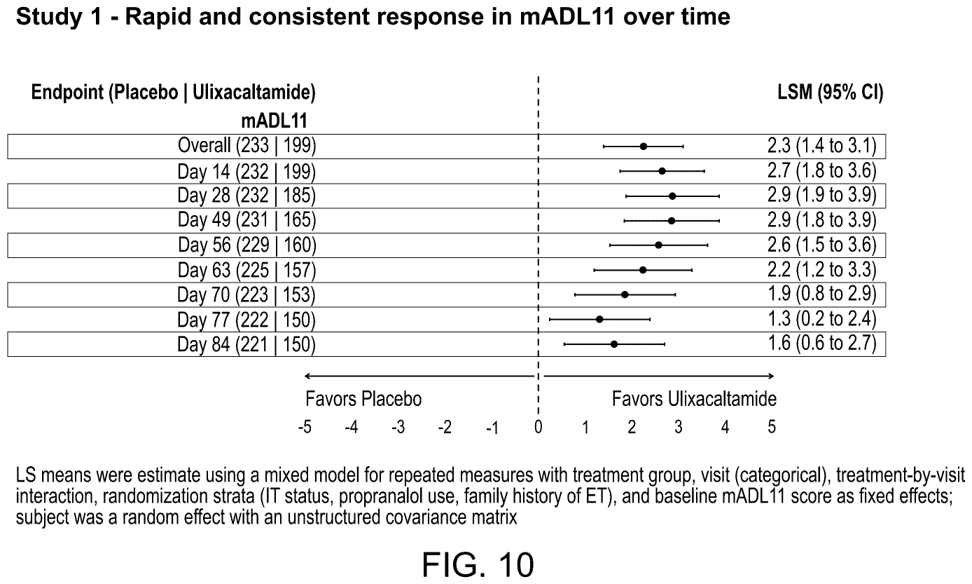

FIG. 10 is a graph illustrating a rapid and consistent response to ulixacaltamide administration through Day 84 in Study 1 as measured by change in mADL11 scores.

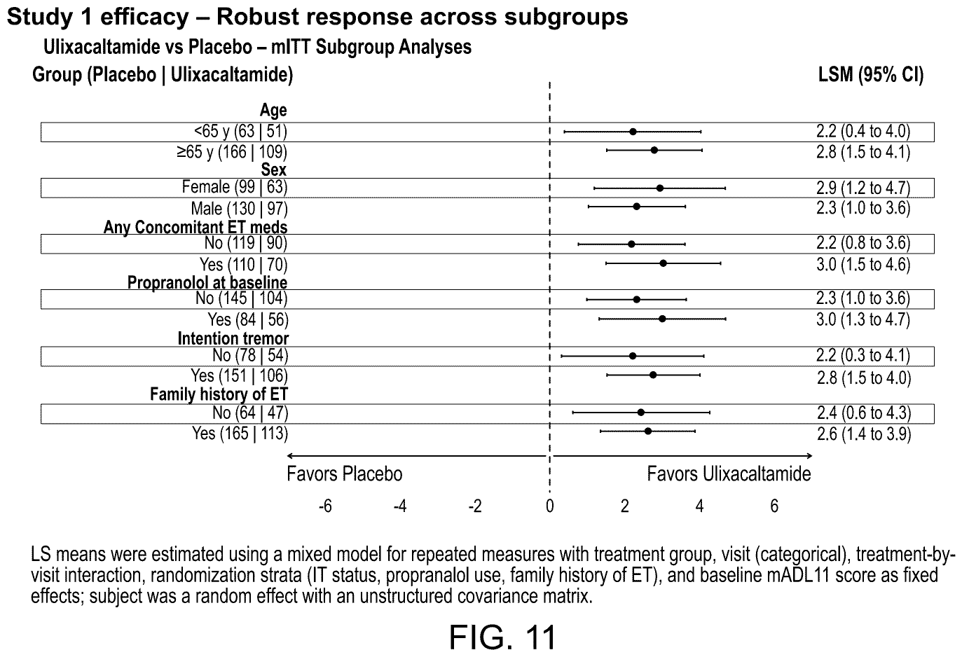

FIG. 11 is a graph illustrating a robust response to ulixacaltamide administration in Study 1 across different participant subgroups.

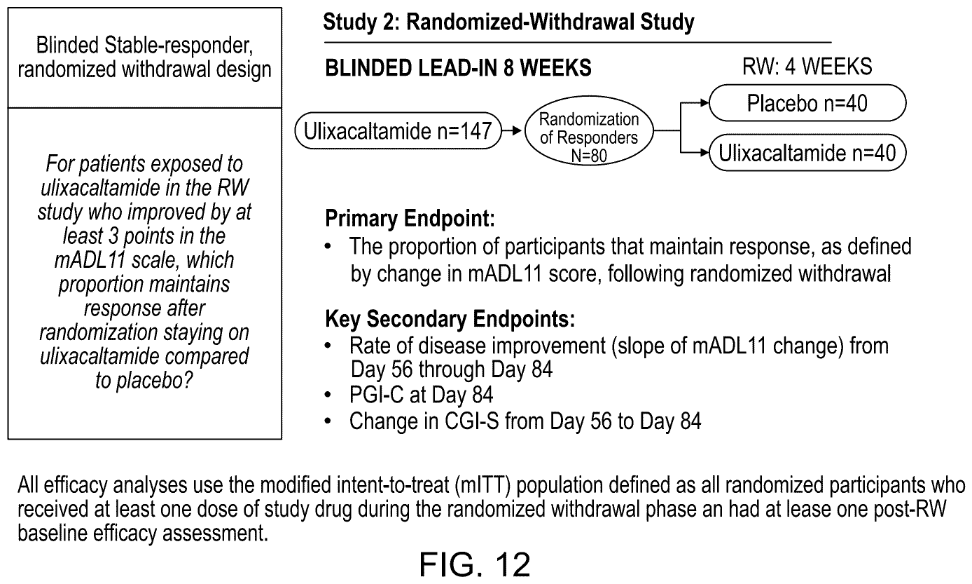

FIG. 12 is a schematic illustrating the analysis of the Study 2 data. This analysis illustrates that for patients exposed to ulixacaltamide in the randomized withdrawal (RW) study who improved by at least 3 points in the mADL11 scale, the proportion of those patients who maintained response after randomication staying on ulixacaltamide as compared to placebo.

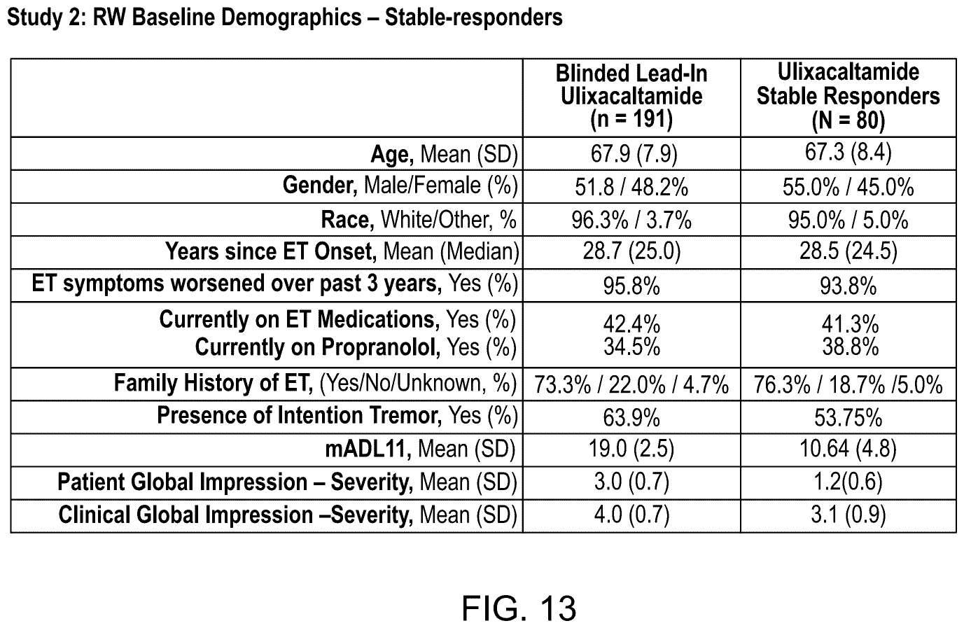

FIG. 13 is a table describing baseline demographics of the participants of Study 2.

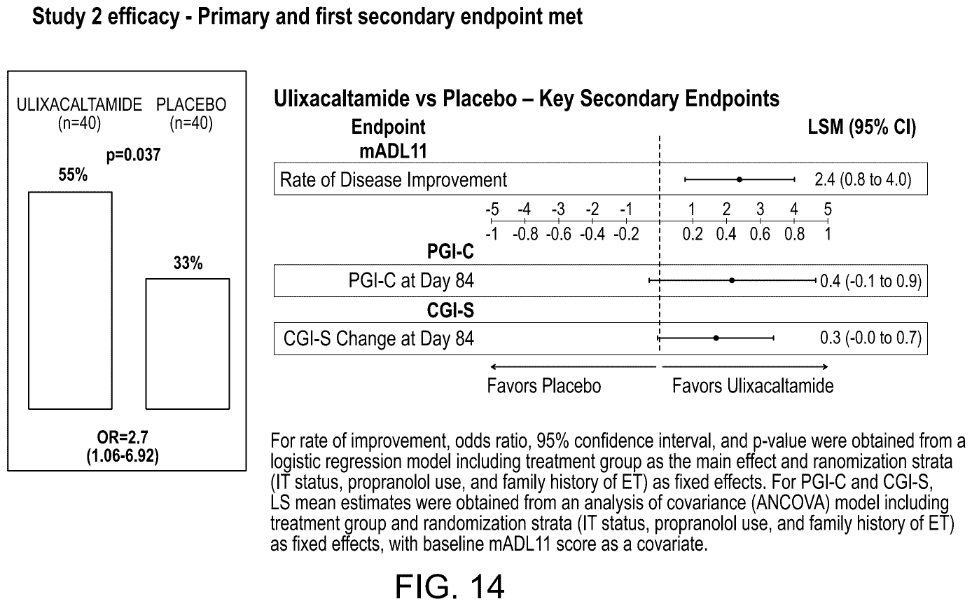

FIG. 14 is a series of graphs illustrating efficacy of ulixacaltamide in Study 2. The bar graph on the left in FIG. 14 shows that 55% of participants that were administered ulixacaltamide (n=40) and were stable responders, and 33% of participants that were administered placebo (n=40) maintained response, as defined by change in mADL11 score, following randomized withdrawal. The graph on the right in FIG. 14 also shows the results of LSM analysis, as it relates to secondary endpoints (rate of disease improvement from Day 56 through Day 84; PGI-C at day 84 and change in CGI-S from day 56 to Day 84) in placebo population (n=40) and ulixacaltabmide population (n=40) in Study 2, and favors ulixacaltamide. The LSM analysis demonstrates that the results favor ulixacaltamide.

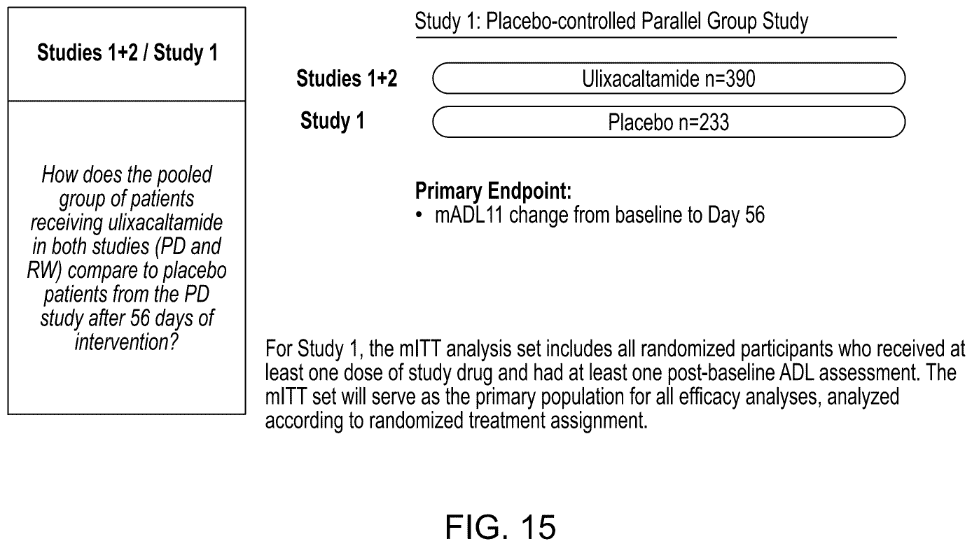

FIG. 15 is a schematic illustrating the analysis of the Study 1 and Study 2 data. This analysis shows how the pooled group of patients receiving ulixacaltamide in both Study 1 and Study 2 and placebo patients from Study 1 after 56 days of intervention.

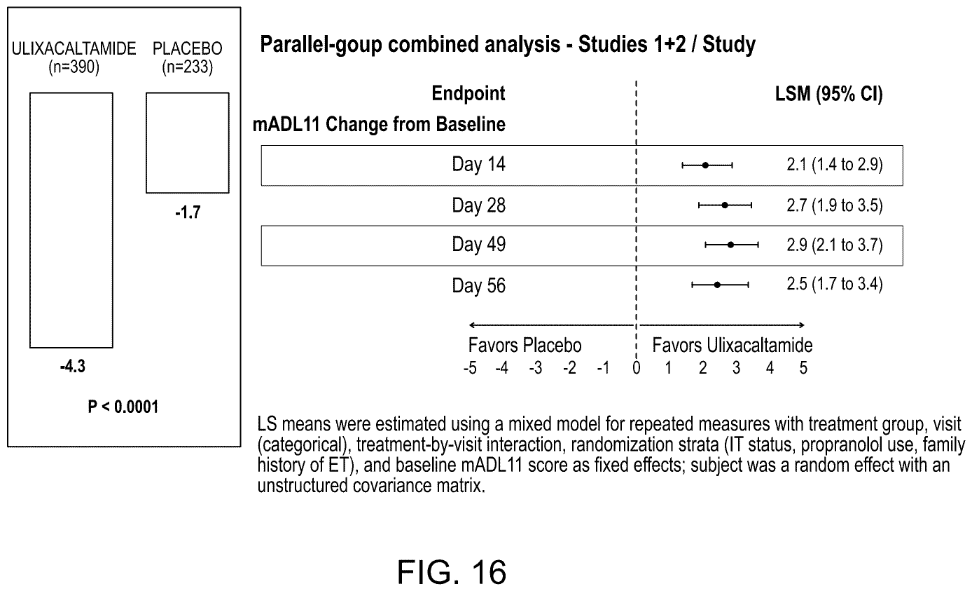

FIG. 16 is a series of graphs illustrating results of the analysis shown in FIG. 15. The bar graph on the left in FIG. 16 shows who participants that were administered ulixacaltamide in Study 1 and Study 2 (n=390) experienced a decrease in mADL11 score of 4.3 points, while the participants that were administered placebo in Study 1 (n=233) experienced a decrease in mADL11 score of 1.7 points. The graph on the right in FIG. 16 shows the results of LSM analysis as it relates to changes in primary endpoint (mADL11 change from baseline) at Day 14, Day 28, Day 49 and Day 56 in placebo population (n=233) and ulixacaltamide population (n=390). The LSM analysis demonstrates that the results favor ulixacaltamide.

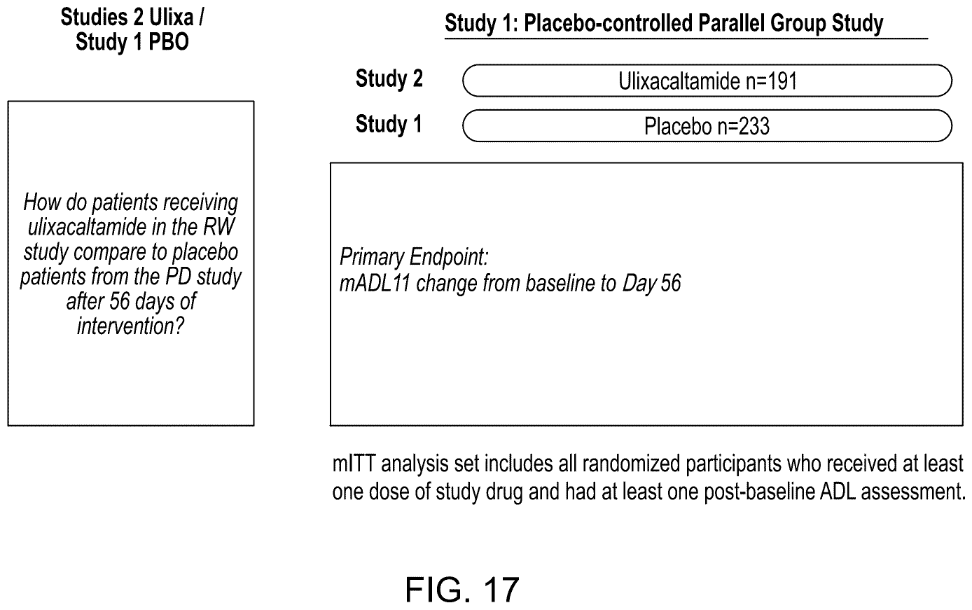

FIG. 17 is a schematic illustrating the analysis of the Study 1 and Study 2 data. This analysis illustrates how patients receiving ulixacaltamide in the RW study compare to patients from the PD study after 56 days of intervention.

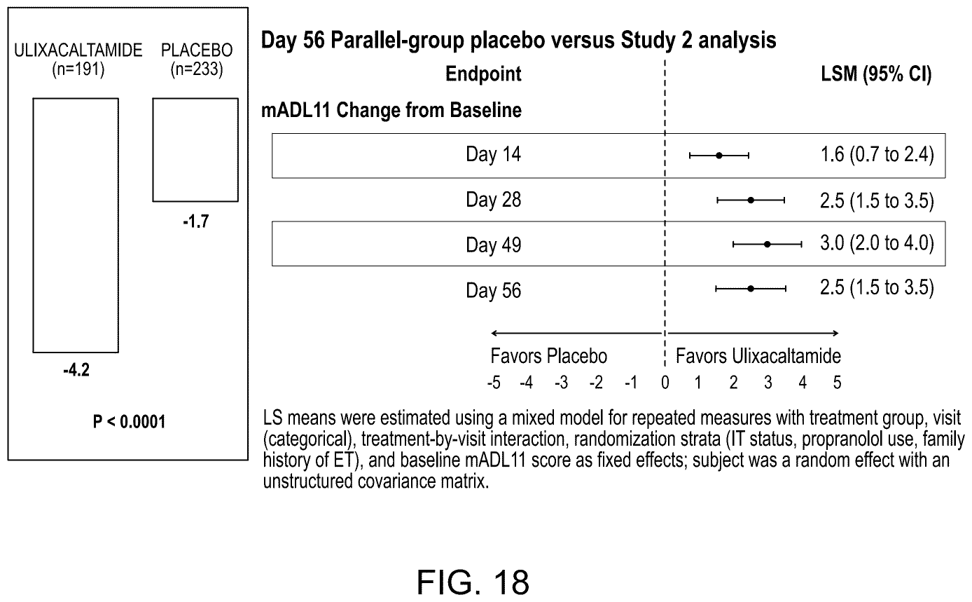

FIG. 18 is a series of graphs illustrating efficacy of ulixacaltamide based on the analysis illustrated in FIG. 17. The bar graph on the left in FIG. 18 shows that participants who were administered ulixacaltamide in Study 2 (blinded lead-in, n=191) experienced a decrease in mADL11 score of 4.3 points, while the subjects administered placebo in Study 1 (n=233) experienced a decrease in mADL11 score of 1.7 points. The graph on the right in FIG. 18 shows the results of LSM analysis as it relates to changes in primary endpoint (mADL11 change from baseline) at Day 14, Day 28, Day 49 and Day 56 in placebo population in Study 1 (n=233) and ulixacaltamide population in Study 2 (blinded lean-in, n=191). The LSM analysis demonstrates that the results favor ulixacaltamide.

Safety across Study 1 and Study 2 remained consistent. There was no change in overall safety provide of ulixacaltamide and no new signals identified. Most common TEAEs (>10%) in participants treated with ulixacaltamide were constipation, dizziness, eurphoric mood, brain fog, headache, paraesthesia, insomnia and fatigue. Discontinuations were primarily due to AEs, with most due to dizziness and brain fog. The majority of TEAEs were mild to moderate in severity, and there were no SAEs related to ulixacaltamide.

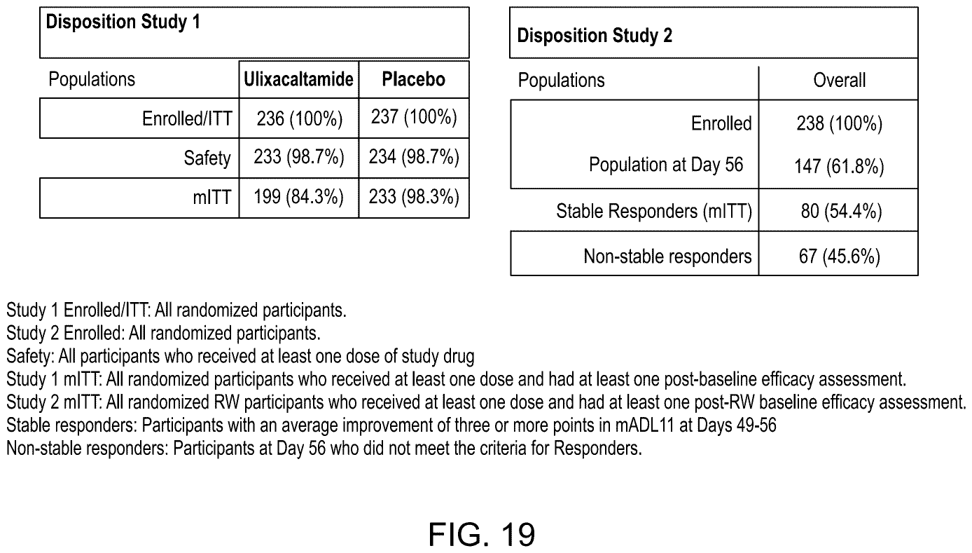

FIG. 19 shows two tables describing disposition of participants in Study 1 and Study 2.

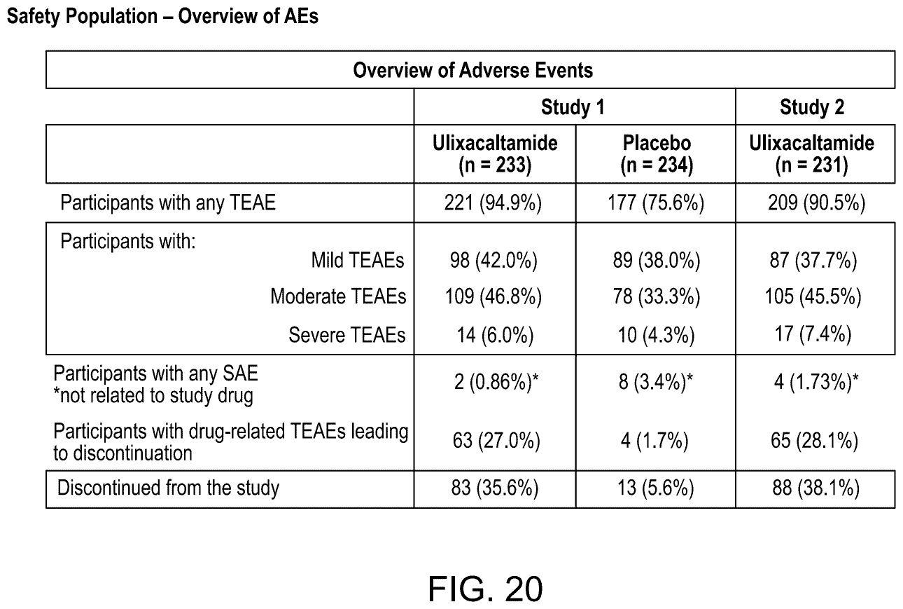

FIG. 20 shows a table with an overview of adverse events in the safety population.

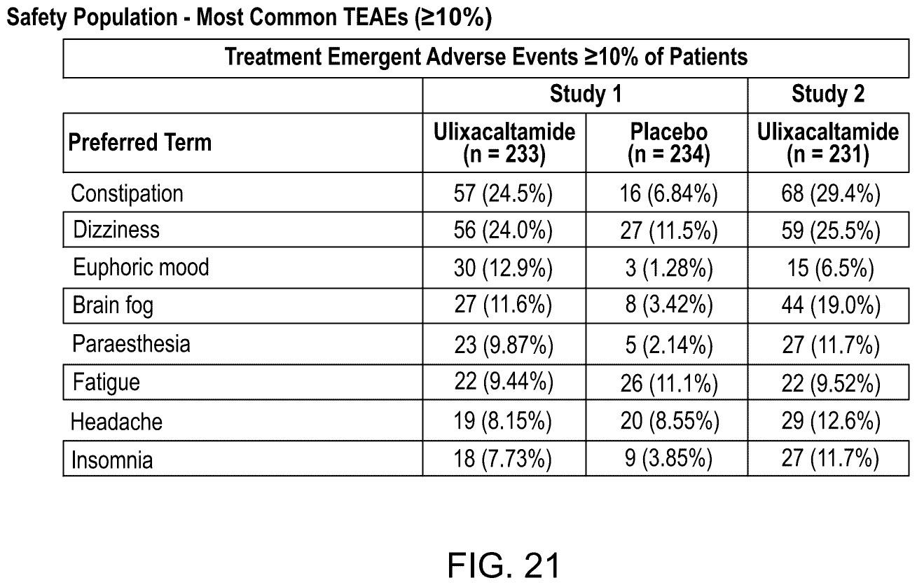

FIG. 21 shows a table with an overview of most common TEAEs (>10%) in the safety population.

The topline results presented in FIGS. 6-21 demonstrate that ulixacaltamide can effectively treat essential tremor.

### Example 2. Essential Tremor Management and Impact: An Online Survey Capturing HCP and Patient Perspectives of Disease Burden

Healthcare Professional (HCP) and patient online surveys were conducted. Only neurologists treating essential tremor and patients with an essential tremor diagnosis were eligible to participate. The HCP survey included 152 neurologists, and 411 patients were invited to participate in the online survey. Two separate patient online surveys were completed and included 150 adults living with essential tremor, and a further 261 adults living with essential tremor who were pre-screened, but did not qualify for the Phase 3 trial described in Example 1.

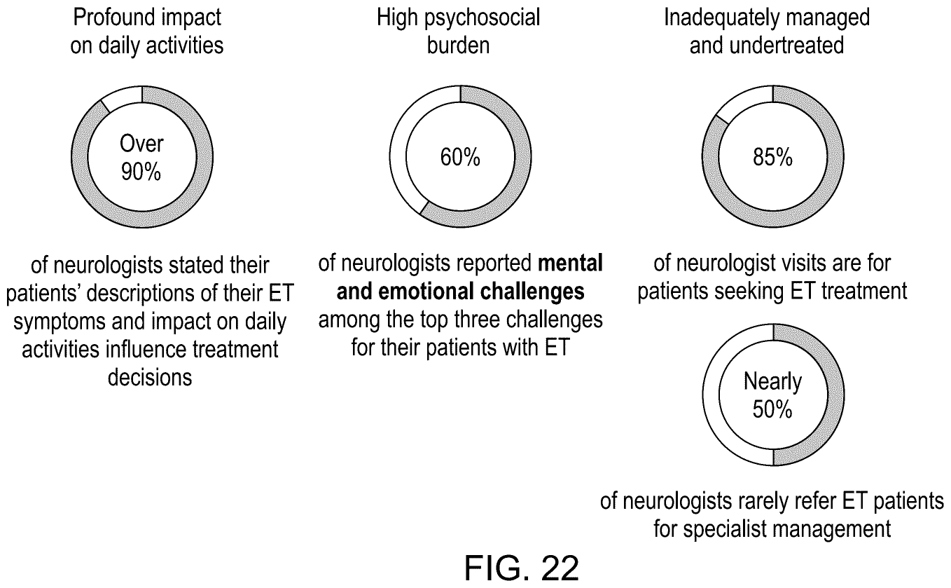

FIG. 22 is a series of schematics illustrating the results of an essential tremor neurologist survey. In the neurologist cohort, the majority reported practicing in academic centers (>75%). Approximately 85% of neurologist visits are for patients seeking treatment. Neurologists noted their patients' primary challenges as tremor symptoms and impairments in activities of daily living with over 90% of neurologists stating that essential tremor's impact on daily living and its symptoms influence their medical treatment decisions. Neurologists reported confidence in managing essential tremor, and nearly half of neurologists reported rarely referring patients for specialist management.

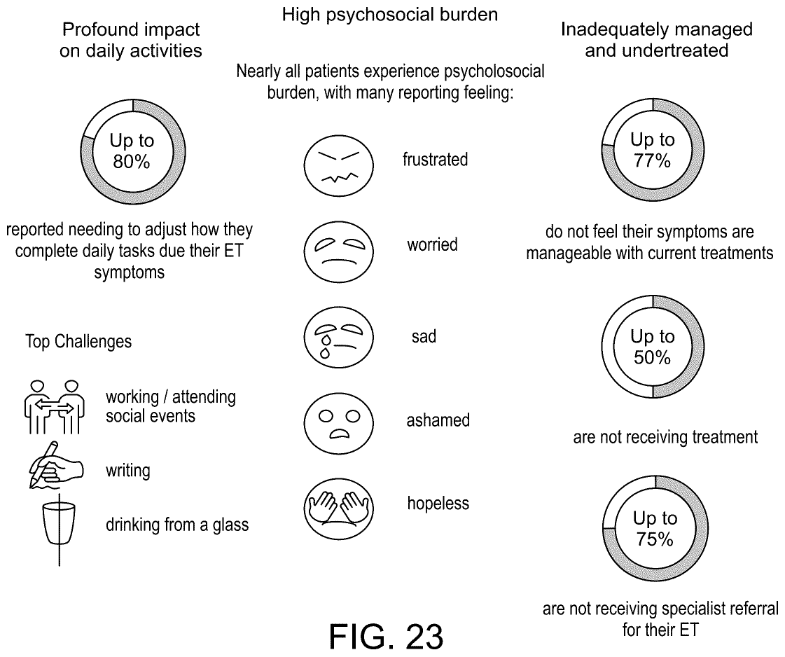

FIG. 23 is a series of schematics illustrating the results of essential tremor patient surveys. Patient responders across the two separate surveys were generally 30-75 years olds who primarily see neurologists and PCPs for their essential tremor management. Patient responders indicated widespread challenges with typical activities of daily living, with up to 80% noting the need to adjust how they complete daily tasks due to their essential tremor symptoms. Almost all reported a level of psychosocial impact, with many reporting feeling frustrated, ashamed, worried, sad and hopeless due to their symptoms. While treatment regimens are diverse, with many patients on multiple interventions, up to 50% reported they are not receiving any treatment. Up to 77% of patients reported not feeling their symptoms are manageable with current treatments. Surveys of >400 essential tremor patients highlight ongoing hidden burden in essential tremor and associated challenges in managing every day life.

### CONCLUSIONS

The findings emphasize the need for more effective, well-tolerated treatments for essential tremor, and the importance of honest physician-patient dialogue. Notably, the surveys highlight the profound impact on daily activities among patients living with essential tremor and associated widespread psychosocial burden, which may be compounded by a degree of normalization of essential tremor impact.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating essential tremor, said method comprising administering to a subject in need thereof compound of formula (I): 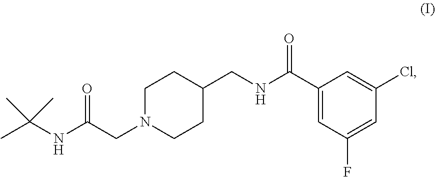 [SMILES: CC(C)(C)NC(=O)CN1CCC(CNC(=O)c2cc(F)cc(Cl)c2)CC1]
or a pharmaceutically acceptable salt thereof,
wherein the compound of formula (I), or a pharmaceutically acceptable salt thereof, is administered to the subject once daily at a dose of about 20 mg to about 60 mg;
such that the modified Activities of Daily Living scale (mADL11) performance score of said subject is decreased by at least about 3 points following administration of the compound of formula (I), or a pharmaceutically acceptable salt thereof, as compared to the mADL11 performance score of said subject prior to the administration of the compound of formula (I), or a pharmaceutically acceptable salt thereof.

2. The method of claim 1, wherein the modified Activities of Daily Living scale (mADL11) performance score of said subject is decreased by at least about 3.5 points following administration of the compound of formula (I), or a pharmaceutically acceptable salt thereof, as compared to the mADL11 performance score of said subject prior to the administration of the compound of formula (I), or a pharmaceutically acceptable salt thereof.

3. The method of claim 1, wherein the modified Activities of Daily Living scale (mADL11) performance score of said subject is decreased by at least about 4 points following administration of the compound of formula (I), or a pharmaceutically acceptable salt thereof, as compared to the mADL11 performance score of said subject prior to the administration of the compound of formula (I), or a pharmaceutically acceptable salt thereof.

4. The method of claim 1, wherein the compound of formula (I), or a pharmaceutically acceptable salt thereof, is administered to said subject at a dose of about 60 mg.

5. The method of claim 1, wherein the compound of formula (I), or a pharmaceutically acceptable salt thereof, is administered to said subject following an initial titration period.

6. The method of claim 5, wherein the compound of formula (I), or a pharmaceutically acceptable salt thereof, is administered to said subject at a dose of about 60 mg following an initial titration period.

7. The method of claim 1, wherein the compound of formula (I), or a pharmaceutically acceptable salt thereof, is administered to the subject for at least 8 weeks.

8. The method of claim 1, wherein said performance score of said subject is decreased by at least about 3 points, at least about 3.5 points, at least about 4 points, at least about 4.5 points, at least about 5 points, at least about 5.5 points, at least about 6 points, at least about 6.5 points, at least about 7 points, at least about 7.5 points, at least about 8 points, at least about 8.5 points, at least about 9 points, at least about 9.5 points or at least about 10 points following administration of the compound of formula (I), as compared to the mADL11 score of said subject prior to the administration of the compound of formula (I).

9. The method of claim 1, wherein a subscore for at least one item assessed as a part of the mADL11 score is decreased.

10. The method of claim 9, wherein said at least one item is selected from the group consisting of: item 1 (speaking); item 2 (feeding with a spoon); item 3 (drinking from a glass); item 4 (hygiene); item 5 (dressing); item 6 (pouring); item 7 (carrying); item 8 (using keys); item 9 (writing); item 10 (working); and item 11 (overall disability).

11. The method of claim 1, wherein said subject is a human.
